(19) United States
(12) Patent Application Publication
(10) Pub. No.: US 2016/0166822 A1
(43) Pub. Date: Jun. 16, 2016

(54) LOCKING DEVICE FOR A Y-PORT FEEDING ADAPTER

(71) Applicant: SECURE GI CONNECTIONS, LLC, Duncansville, PA (US)

(72) Inventors: Alan D. Dodson, Altoona, PA (US); Mary K. Dodson, Altoona, PA (US)

(73) Assignee: SECURE GI CONNECTIONS, LLC, Duncansville, PA (US)

(21) Appl. No.: 14/568,399

(22) Filed: Dec. 12, 2014

(51) Int. Cl. A61M 39/10

(52) U.S. Cl. CPC A61M 39/10; A61M 39/1055

(57) ABSTRACT

A locking device for medical tube coupling includes an elongated body positionable during use adjacent an exterior surface of the medical tube coupling, a first flange carried on one end of the body, a slot formed through a thickness of the first flange in an open communication with an edge surface thereof, and a second flange carried on a longitudinally opposite end of the body and extending in a direction of the first flange. The second flange may include the same slot and/or a cavity in an inner surface thereof. The locking device may be made adjustable to lock a connection between a tube and the medical tube coupling and/or securely cap the inlet port of the medical tube coupling when tube is removed. The device may also include a third flange being disposed on the elongated body between the first and second flanges.

### CROSS-REFERENCE TO RELATED APPLICATIONS

N/A

### STATEMENT REGARDING FEDERALLY SPONSORED RESEARCH AND DEVELOPMENT

N/A

### REFERENCE TO SEQUENCE LISTING, A TABLE, OR A COMPUTER PROGRAM LISTING COMPACT DISC APPENDIX

N/A

### BACKGROUND OF THE INVENTION

1. Technical Field

The subject matter relates to medical tube coupling. It further relates to locking a connection between a medical tube and a coupling member and/or closing or capping a medical tube receiving end of the coupling member when the medical tube is removed.

2. Description of Related Art

Tubular administration for administering oxygen, medical fluids or feeds and/or evacuation of fluids from a patient requires a connector or a coupling between different tubular members. Such tubular administration may be related to evacuating or draining urine into a drainage bag. Such tubular administration may be related to enteral feeding delivering a nutritionally complete feed, containing proteins, carbohydrates, fats, water, minerals and vitamins, directly into the stomach, duodenum or jejunum. When tubular administration is related to enteral feeding, it requires a catheter or a tube inserted into a patient through an abdominal wall and having an exposed end thereof either inserted into an outlet port of a Y-port feeding adapter or being manufactured integral therewith. One inlet port of the Y-port feeding adapter is configured to receive a feeding tube and is closed or capped with a removable cap and plug member during use when the feeding tube is disconnected. The connection between the feeding tube and Y-port feeding adapter may come apart during use, as well as the integral cap and plug member may disengage from the feeding tube inlet port of the Y-port feeding adapter resulting in unintentional fluid leakage. Other types of medical tube couplings have also been associated, to the best knowledge of Inventors, with unintentional fluid leakage issues.

Therefore, there is a need to provide a secure locking connection between an inlet tube and a medical tube coupling and/or securely close or cap the inlet port of the medical tube coupling when inlet tube is not in use.

### BRIEF DESCRIPTION OF THE DRAWINGS

The accompanying drawings are incorporated in and constitute part of the specification and illustrate various embodiments. In the drawings:

FIG. 1 illustrates a perspective view of one exemplary device configured and operable to lock a connection between a feeding tube and an inlet port of a medical tube coupling or close or cap an inlet port of the medical tube coupling;

FIG. 2 illustrates an end view of the device in FIG. 1;

FIG. 3 illustrates a perspective view of one example of the device in FIG. 1;

FIG. 3A illustrates a partial perspective view of another example of the device in FIG. 1;

FIG. 4 illustrates a partial perspective view of a further example of the device in FIG. 1;

FIG. 5 illustrates yet another perspective view of the device in FIG. 1;

FIG. 6 illustrates one partial elevation view of the device in FIG. 1;

FIG. 7 illustrates another partial elevation view of the device in FIG. 1;

FIG. 8 illustrates yet another partial elevation view of the device in FIG. 1;

FIG. 9 illustrates a further partial elevation view of the device in FIG. 1;

FIG. 10 illustrates a perspective environmental view of the device of FIG. 1 in a combination with the medical tube coupling;

FIG. 11 illustrates a perspective view of another exemplary device configured and operable to close or securely cap an inlet port of the medical tube coupling;

FIG. 12 illustrates another perspective view of the device in FIG. 11;

FIG. 13 illustrates a perspective environmental view of the device of FIG. 11 in a combination with the medical tube coupling;

FIG. 14 illustrates a perspective view of another exemplary device configured and operable to lock a connection between a feeding tube and an inlet port of a medical tube coupling or close or cap an inlet port of the medical tube coupling;

FIG. 14A illustrates a perspective view of an alternative device of FIG. 14 configured and operable to lock a connection between a feeding tube and an inlet port of a medical tube coupling or close or securely cap the inlet port of the medical tube coupling;

FIG. 15 illustrates a front view of a further exemplary device configured and operable to either lock a connection between a feeding tube and an inlet port of a medical tube coupling or close or securely cap the inlet port of the medical tube coupling when the feeding tube is removed therefrom;

FIG. 16 illustrates a perspective view of yet another exemplary device configured to lock a connection between a feeding tube and an inlet port of a medical tube coupling or close or securely cap the inlet port of the medical tube coupling and shown as operable to lock the connection between the feeding tube and the inlet port;

FIG. 17 illustrates another perspective view of the device in FIG. 16 and shown as operable to directly abut and securely cap the inlet port of the medical tube coupling when the feeding tube is removed from the inlet port;

FIG. 18 illustrates an exploded perspective view of a further exemplary device configured and operable to lock a connection between a feeding tube and an inlet port of a medical tube coupling or close or securely cap the inlet port of the medical tube coupling;

FIG. 19 illustrates another perspective view of the device in FIG. 18;

FIG. 20 illustrates a top view of yet a further exemplary device configured and operable to lock a connection between a feeding tube and an inlet port of a medical tube coupling or close or securely cap an inlet port of the medical tube coupling;

FIG. 21 illustrates a cross-sectional longitudinal view of the device along lines 21-21 in FIG. 20;

FIG. 22, labeled “Prior Art”, illustrates a medical tube coupling with the inlet tube and fitting inserted into an inlet port;

FIG. 23, labeled “Prior Art”, illustrates a medical tube coupling with the inlet tube and fitting removed and the inlet port being plugged with the integral cap or plug;

FIG. 24 is another perspective view of the exemplary device configured and operable to lock a connection between a feeding tube and an inlet port of a medical tube coupling or close or securely cap the inlet port of the medical tube coupling;

FIG. 25 is a top plan view of the device in FIG. 24;

FIG. 26 is a bottom plan view of the device in FIG. 24;

FIG. 27 is a front elevation view of the device in FIG. 24, rear elevation view is identical;

FIG. 28 is a right side elevation view of the device in FIG. 24, left side elevation view is identical;

FIG. 29 is a perspective environmental view of the device in FIG. 24 in a combination with medical tube coupling illustrated as a Y-port feeding adapter;

FIG. 30 is another perspective view of the exemplary device configured and operable to lock a connection between a feeding tube and an inlet port of a medical tube coupling or close or securely cap the inlet port of the medical tube coupling;

FIG. 31 is a top plan view of the device in FIG. 30;

FIG. 32 is a bottom plan view of the device in FIG. 30;

FIG. 33 is a front elevation view of the device in FIG. 30, rear elevation view is identical;

FIG. 34 is a right side elevation view of the device in FIG. 30, left side elevation view is identical;

FIG. 35 is a perspective environmental view of a device in FIG. 30 in a combination with a medical tube coupling illustrated as a Y-port feeding adapter;

FIG. 36 is another perspective view of the exemplary device configured and operable to lock a connection between a feeding tube and an inlet port of a medical tube coupling or close or securely cap the inlet port of the medical tube coupling;

FIG. 37 is a top plan view of the device in FIG. 36;

FIG. 38 is a bottom plan view of the device in FIG. 36;

FIG. 39 is a front elevation view of the device in FIG. 36, rear elevation view is identical;

FIG. 40 is a right side elevation view of the device in FIG. 36, left side elevation view is identical;

FIG. 41 is a perspective environmental view of the device in FIG. 36 in a combination with a medical tube coupling illustrated as a Y-port feeding adapter;

FIG. 42 is another perspective view of the exemplary device configured and operable to close or securely cap the inlet port of the medical tube coupling;

FIG. 43 is a top plan view of the device in FIG. 42;

FIG. 44 is a bottom plan view of the device in FIG. 42;

FIG. 45 is a front elevation view of the device in FIG. 42, rear elevation view is identical;

FIG. 46 is a right side elevation view of the device in FIG. 42;

FIG. 47 is a left side elevation view of the device in FIG. 42;

FIG. 48 is a perspective environmental view of the device in FIG. 42 in a combination with a medical tube coupling illustrated as a Y-port feeding adapter;

FIG. 49 is another perspective view of the exemplary device configured and operable to close or securely cap the inlet port of the medical tube coupling;

FIG. 50 is a top plan view of the device in FIG. 49;

FIG. 51 is a bottom plan view of the device in FIG. 49;

FIG. 52 is a front elevation view of the device in FIG. 49, rear elevation view is identical;

FIG. 53 is a right side elevation view of the device in FIG. 49;

FIG. 54 is a left side elevation view of the device in FIG. 49;

FIG. 55 is a perspective environmental view of the device in FIG. 49 in a combination with a medical tube coupling illustrated as a Y-port feeding adapter;

FIG. 56 is another perspective view of the exemplary device configured and operable to close or securely cap the inlet port of the medical tube coupling;

FIG. 57 is a top plan view of the device in FIG. 56;

FIG. 58 is a bottom plan view of the device in FIG. 56;

FIG. 59 is a front elevation view of the device in FIG. 56, rear elevation view is identical;

FIG. 60 is a right side elevation view of the device in FIG. 56;

FIG. 61 is a left side elevation view of the device in FIG. 56; and

FIG. 62 is a perspective environmental view of the device in FIG. 56 in a combination with a medical tube coupling illustrated as a Y-port feeding adapter.

### DETAILED DESCRIPTION OF EXEMPLARY EMBODIMENTS

Prior to proceeding to the more detailed description of the present invention, it should be noted that, for the sake of clarity and understanding, identical components which have identical functions have been identified with identical reference numerals throughout the several views illustrated in the drawing figures.

The following detailed description is merely exemplary in nature and is not intended to limit the described examples or the application and uses of the described examples. As used herein, the words “example”, “exemplary” or “illustrative” means “serving as an example, instance, or illustration.” Any implementation described herein as “example”, “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure, which is defined by the claims. For purposes of description herein, when employed, the terms “upper,” “lower,” “left,” “rear,” “right,” “front,” “vertical,” “horizontal,” “exterior,” “interior,” and derivatives thereof shall relate to the invention as oriented in the Figures. Furthermore, there is no intention to be bound by any expressed or implied theory presented in the preceding technical field, background, or the following detailed description. It is also to be understood that the specific devices and processes illustrated in the attached drawings, and described in the following specification, are simply examples of the inventive concepts defined in the appended claims. Hence, specific dimensions and other physical characteristics relating to the examples disclosed herein are not to be considered as limiting, unless the claims expressly state otherwise.

Furthermore, term “coupling” or “medical tube coupling” is intended to mean herein, without limitation, a connector, a catheter, a feeding adapter for interal feeding, a Y-port feeding adapter for interal feeding, a catheter used in administering intravenous (IV) fluid to a patient, a combination of a catheter and a male IV connector used in administering IV fluids, a connection or a connecting member between breathing or respiratory tubing members and/or a connection or a connecting member between a urinary tubing and a drainage bag.

The particular embodiments of the present disclosure generally provide devices and methods directed to securely locking a connection between an inlet tube and an inlet port of a medical tube coupling and/or closing or securely capping the inlet port of the medical tube coupling.

In particular embodiments, a device or an article of manufacture is provided of a unitary, one-piece construction.

In particular embodiments, a device or an article of manufacture is provided as an assembly of two or more components.

In particular embodiments, a device or an article of manufacture is provided to only lock a connection between a tube and an inlet port of a medical tube coupling.

In particular embodiments, a device or an article of manufacture is provided to only close or securely cap an inlet port of a medical tube coupling.

In particular embodiments, a device or an article of manufacture is provided to lock, improve or secure a connection between a tube and an inlet port of a medical tube coupling and/or closing or securely capping the inlet port of the medical tube coupling.

The forgoing exemplary embodiments are illustrated in a combination with a Y-port feeding adapter for enteral feeding.

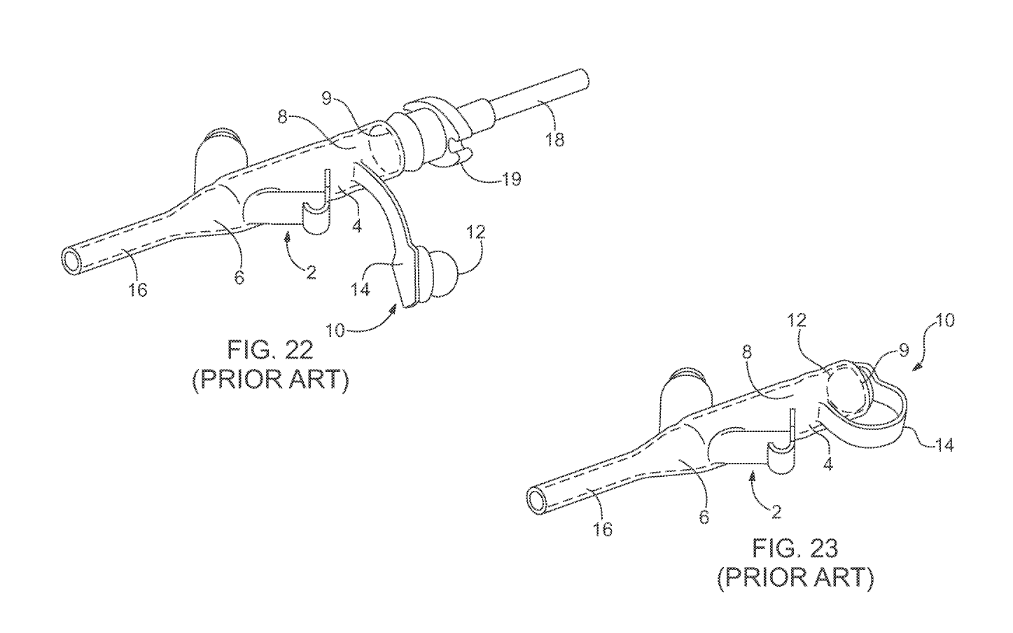

Briefly, for the sake of the reader's convenience, the Y-port feeding adapter 2 for enteral feeding is best illustrated in FIGS. 22-23 as having a body 4, an outlet conduit 6 with an outlet port 7 at one end of the body 4, an inlet conduit 8 with an inlet port 9 at an axially opposite end of the body 4 and a movable cap 10 having a male plug 12 and being permanently attached to the body 4 with a tether or strap member 14 having one end thereof being integral with the body 4 so that the movable cap 10 is always attached to the body 4 during use of the Y-port feeding adapter 2. Also partially shown are an outlet tube 16 transmitting fluid or feed from the outlet port 7 of the Y-port feeding adapter 2 and an inlet or feeding tube 18 which is connected to a fitting 19 and transmitting fluid or feed into the inlet port 9 of the the Y-port feeding adapter 2. The male plug 12 can be of any conventional construction and may be also replaced by a female plug sized to receive an exterior surface of the inlet conduit 8.

FIG. 22, labeled “Prior Art” illustrates the Y-port feeding adapter 2 with the inlet tube 18 and fitting 19 inserted into the inlet port 9, while FIG. 23, also labeled “Prior Art”, illustrates the Y-port feeding adapter 2 with the inlet tube 18 and its fitting 19 removed and the inlet port 9 being plugged with plug 12 of the integral cap 10. Generally, the inlet tube 18 may be removed when the user completed the feeding effort. The plug 12, shown as a male plug, may be provided as a female plug with a cavity sized to receive the exterior surface of the inlet port 9. It is also contemplated herewithin that the Y-port feeding adapter 2 may be provided without the integral cap 10.

The term “securely” is intended to mean herein “in a manner that prevents unintentional or unintended disengagement of the device from the direct engagement with the inlet port 9 sufficient to prevent unintentional or unintended leakage therefrom or unintentional or unintended disengagement of the device from the engagement with an exterior surface of the movable cap 10 closing such inlet port 9 so as to prevent unintentional or unintended disengagement of the plug 12 from the inlet port 9 and subsequent leakage therefrom”.

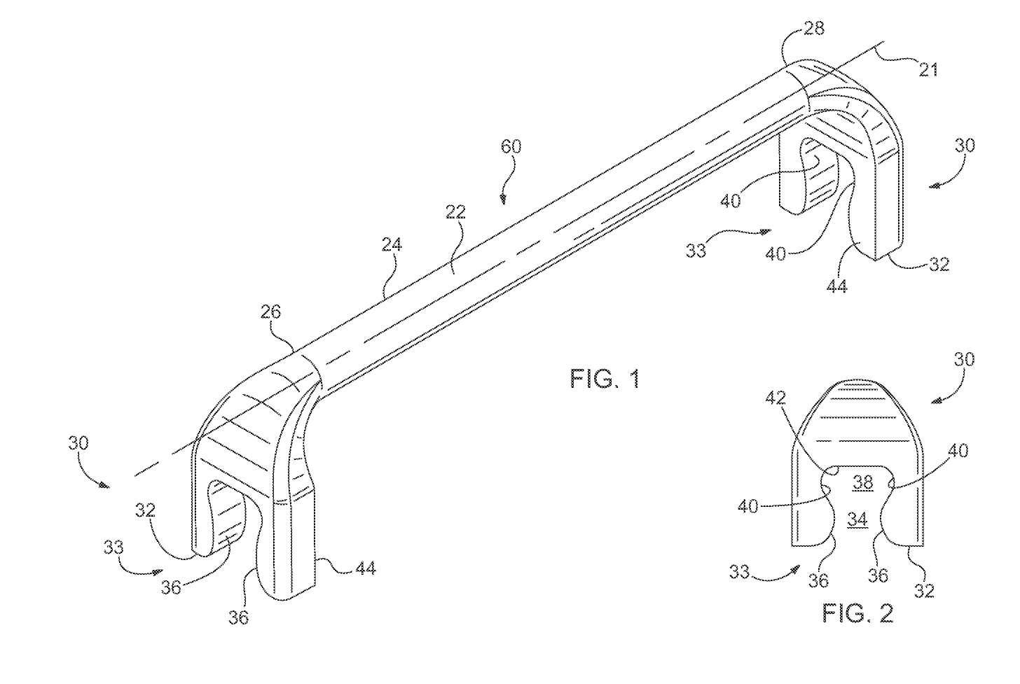

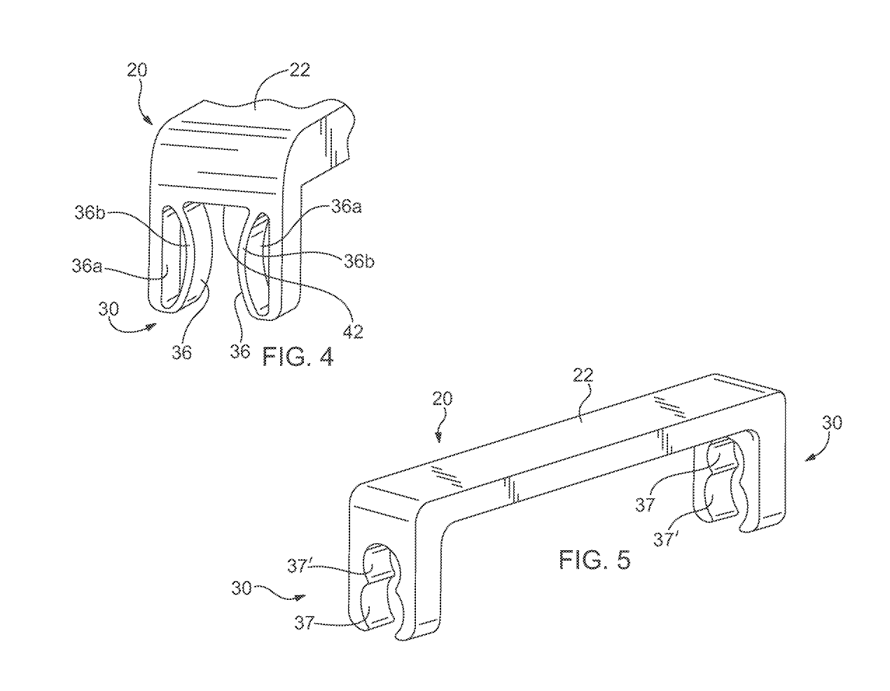

In one exemplary embodiment of FIGS. 1-10, the device or the article of manufacture 20 is provided to only lock a connection between a feeding tube 18 having the fitting 19 and an inlet port 9 of a Y-port feeding adapter 2 and includes an elongated body 22 with an exterior surface 24 and a pair of a longitudinally opposite ends 26 and 28. The terms “lock” or “locking” is intended to mean herein “to prevent unintentional or unintended separation of the fitting 19 and/or inlet tube 18 from the inlet port 9”. The elongated body 22 also defines a longitudinal axis 21 of the device 20. The shape of exterior surface 24 may be defined by one or more curve portions, one or more straight portions and any combinations thereof. Thus, the elongated body 22, illustrated in FIG. 1 as having a uniform round cross-sectional shape in a plane normal to a length of the elongated body 22, may be provided with other cross-sectional shapes, including cross-sectional shapes varying in size and/or exterior profile along one or more length portions of the elongated body 22. For example, FIGS. 4-5 illustrate elongated body 22 as having a rectangular cross-section. The elongated body 22 can be any one of solid, hollow and a combination thereof.

The device 20 also includes a pair of retention or locking portions or flanges 30, each extending from a respective end of the elongated body 22. The flanges 30 may extend normal to the length of the elongated body 22 so that the flanges 30 are parallel to each other or they may extend at other angles including extending toward or away from each other. One or both flanges 30 may be flexible, rigid and a combination thereof.

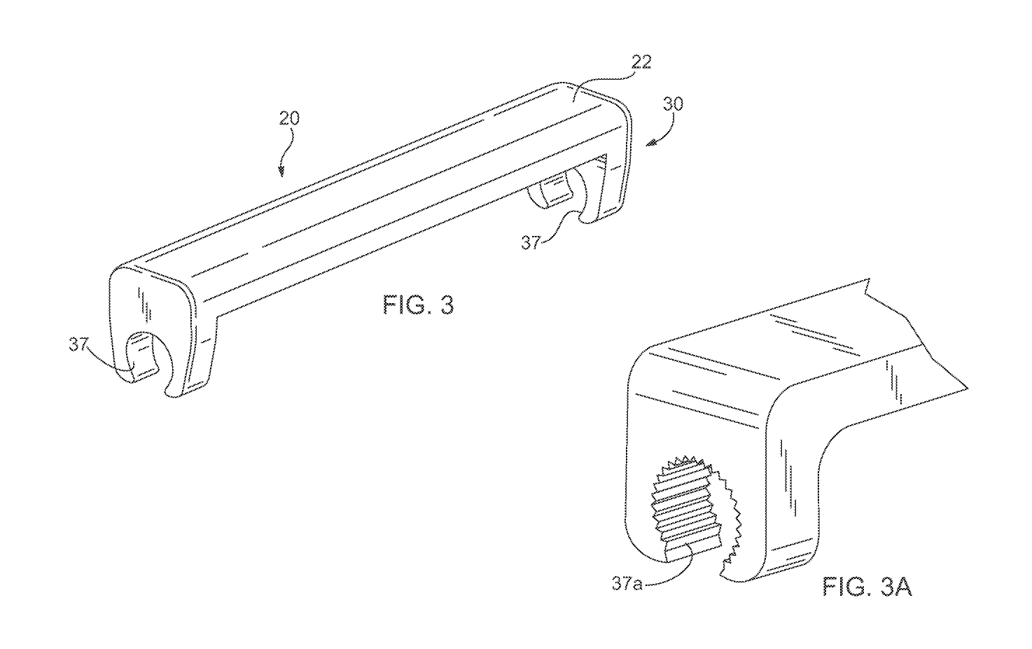

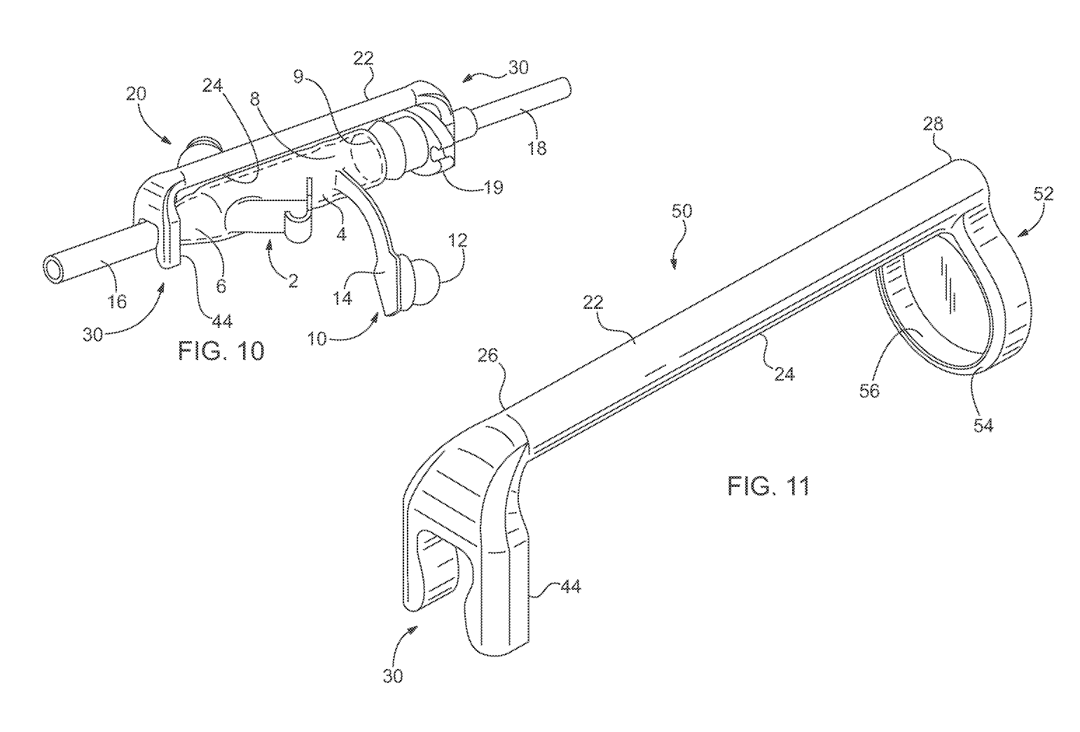

In a further reference to FIGS. 1-2, each flange 30 includes a pair of spaced apart surfaces to define a thickness of the flange 30. The exterior peripheral shape or the contour of the flange 30 is not a limiting feature in this and other exemplary embodiments and can be of any shape. Although the flange 30 is illustrated in various figures as having a pair of parallel planar surfaces, one or more surfaces can have a curved or otherwise non-planar shape and/or one or more surfaces can be inclined at an angle other than ninety (90) degree relative to the length of the elongated body 22. The flange 30 further includes a slot 33 that may be defined by an opening or a passageway 34 formed through the thickness of the flange 30 and terminating, in one example, into a cavity 38 being in an open communication therewith. The opening 34 includes a pair of first surface portions 36 disposed in an open communication with a free end 32 of the flange 30 and extending toward the elongated body 22. The first surface portions 36 may be any one of curved, straight and a combination thereof and are illustrated in the example of FIGS. 1-2 as curving toward each other. The inwardly curved first surface portions 36 may be advantageous in preventing unintended disengagement of the flange 30 from the outlet tube 16 and/or inlet tube 18. The cavity 38 may be defined by a pair of second surface portions 40. The second surface portions 40 may be any one of curved, straight and a combination thereof and are illustrated in the exemplary embodiment of FIGS. 1-2 as curving in directions being opposite to directions of the first surface portions 36. The cavity 38 may also include a straight surface portion 42 or surface portions 40 may continue to meet with each other. During use, the exterior surface 24 of the elongated portion 22 is positioned either in a direct abutting contact or at a distance from an exterior surface of the body 4, while the slot 33 is employed to receive therethrough one of the outlet and inlet tubes, 16 and 18 respectively. In a further reference to FIG. 10, one flange 30 is positioned at the outlet conduit 6 so that the outlet tube 16 is inserted into its respective opening 34 and may be further inserted into the cavity 38, depending on the exterior diameter of the outlet tube 16, while the other flange 30 is positioned at the inlet conduit 8 in an abutting engagement with the fitting 19 of the inlet tube 18 so that the inlet tube 18 is at least inserted into its respective opening 34 and may be further inserted into the cavity 38. The opening 34 and/or the cavity 38 may be sized and shaped to accommodate either a wide range of the exterior diameters (varying exterior diameters) of the outlet and/or inlet tubes, 16 and 18 respectively or may be sized and shaped to accommodate a specific/unique size of such tubes. The outlet and/or inlet tubes, 16 and 18 respectively may be allowed to flatten, either in the opening 34, cavity 38 or a combination of both, without impeding sufficient/adequate fluid or feed passage therethrough. The cavity 38 may be sized and shaped so as to prevent flattening of the outlet and/or inlet tubes, 16 and 18 respectively, received therewithin. It is also contemplated that the tubes of smaller diameters may be positioned within the cavity 38, with or without a full contact with an interior surface thereof, and prevented, by the narrower opening 34, particularly having the curved surfaces 36, from unintentionally exiting the cavity 38, when the exterior diameter of the outlet and/or inlet tubes, 16 and 18 respectively, is smaller than the size of the cavity 38. In another example, the slot 33 may be also provided as a single opening 37 having a C-shaped cross-section in a plane transverse to the longitudinal axis 21, as is best shown in FIG. 3 and may have an irregular surface defined, by way of one example only in FIG. 3A, by ribs 37a or any other shaped projections, that are of a height and a flexibility to accommodate exterior diameters of the tubes 16 and/or 18 of varying sizes. In yet another example of FIG. 4, the slot 33 may be simply defined by the first curved surface portions 36 and may be further defined by apertures 36a so as to form walls 36b that are sufficiently flexible to move away from each other during insertion or removal of the tubes 16 or 18 and are sufficiently resilient to move toward each other to cage, either partially or completely, the tubes 16 or 18 being inserted into the slot 33 or removed from the passageway 34 of the slot 33. In a further example of FIG. 5, the slot 33 may be provided as a pair of openings 37 and 37′, each having a C-shaped cross-section in a plane transverse to the longitudinal axis 21. The inner surface(s) in the examples of FIGS. 4-5 may be also provided with the irregular surface of the example of FIG. 3A.

Likewise, as it will be explained in more details below, the distance between inner surfaces 44 of the opposite flanges 30 is sized to accommodate the length of the Y-port feeding adapter 2 and is sufficient to prevent unintentional detachment of the inlet tube 18 and, more particularly, its fitting 19 from the inlet port 9. It is contemplated that the edge of the slot 33 of the flange 30, positioned at the outlet conduit 6, either abuts an exterior surface of such outlet conduit 6 and exerts force thereonto along the longitudinal axis 21 or the interior surface and/or edge of the slot 33 within such flange 30 clamps, cages and/or abuts the exterior surface of the outlet tube 16 in a manner sufficient to prevent unintended detachment of the flange 30 therefrom. The slot 33 does not have to be sized and shaped so as to provide a snap-fit or clamping engagement between the entire interior surface of the slot 33 and the exterior surface of the outlet conduit 6. In other words, the size and shape of the inner surface of the slot 33 does not have to be substantially identical to the size and shape of the exterior surface of the outlet conduit 6. This may be particularly advantageous when the exterior surface of the outlet conduit 6 is tapered and varies in shape and/or size between different Y-port feeding adapters 2.

The elongated body 22, in this and other exemplary embodiments, does not have to be straight, as illustrated in FIGS. 1-10 and may include at least one of bend, offset, twist and any combination thereof. By way of one example only, the elongated body 22 may be so configured that the flanges 30 are still positioned in a parallel relationship with each other but are rotated from each other about the longitudinal axis 21.

The device 20 may be manufactured from any one of plastic material, metallic material, elastomeric material and any combinations thereof. The device 20 may be provided as a unitary, one-piece construction, for example by a molding or a casting process, or a construction wherein one or both flanges 30 are provided as separate members being either permanently/rigidly or releasably connected to the elongated body 22.

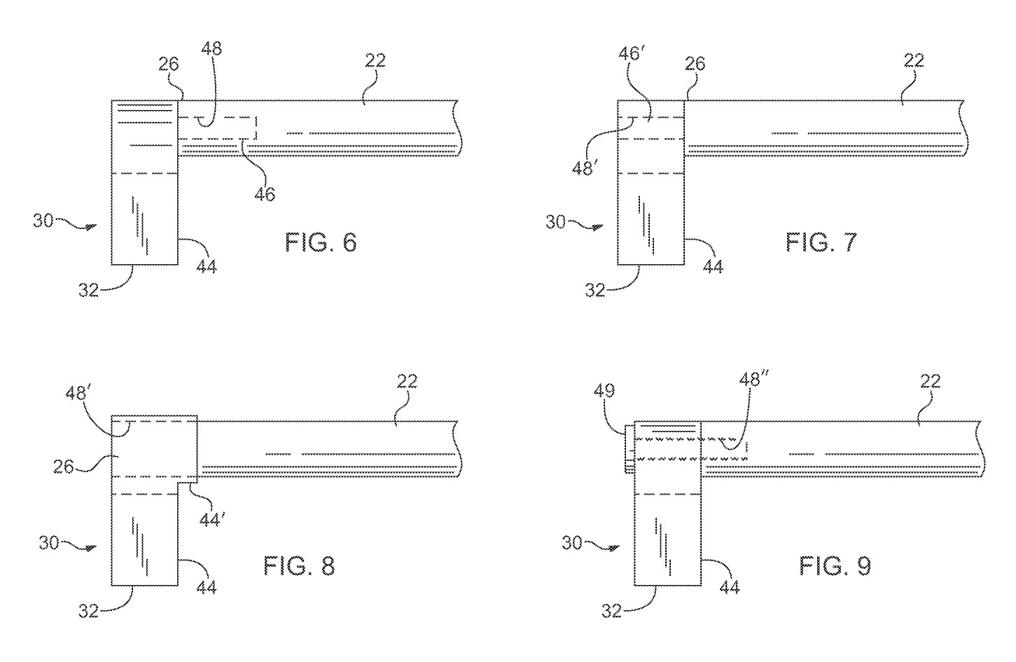

If the flange 30 is to be provided as a separate member and assembled with the elongated body 22, the invention contemplates various means for attaching the flange 30 to the elongated body 22. In one example of FIG. 6, the interior surface 44 of the flange 30 may be adapted with a stem 46 sized to be received, for example by a friction fit, within a bore 48 in a respective end 26 and/or 28 of the elongated body 22. In another example of FIG. 7, the position of the stem 46 and the bore 48 may be reversed, where the stem 46′ is an extension of the elongated body 22 and the bore 48′ is at least formed within the thickness of the flange 30 and even extending therethrough. In a further example of FIG. 8, the flange 30 may be also provided with the bore 48′ sized to receive, for example by a friction fit, the peripheral surface of the end 26 of the elongated body 22 without the stem 46. Also shown in FIG. 8 is that the inner surface 44 may be further adapted with a projection 44′ and wherein the bore 48′ further extends through the projection 44′. In yet another example of FIG. 9, a fastened connection between the flange 30 and the elongated body 22 is also contemplated herein by using a threaded fastener 49. A connection by way of an adhesive (not shown) is also contemplated. The example connections in FIGS. 6-9 provide means for attaching separate flange(s) 30 to the elongated body 22 in either permanent or releaseable manner.

In another exemplary embodiment of FIGS. 11-13, the device or the article of manufacture 50 includes the above described elongated body 22 and one flange 30 as a first retention or locking portion. The device 50 also includes a flange 52, as a second retention or locking portion extending in a direction of the flange 30 from the opposite end of elongated body 22, referenced by a numeral 28. The flange 52 may include a cavity 56 formed in an interior surface 54 thereof, where the interior surface 54 faces the interior surface 44 of the flange 30. The interior surface 54 and/or cavity 56 may be sized and shaped so as to directly abut an end of the inlet conduit 8 or an exterior surface of the movable cap 10 having its plug 12 inserted into the inlet port 9 on the Y-port feeding adapters 2 employing such movable cap 10. When the cavity 56 is provided, for example when the Y-port feeding adapter 2 does not employ the movable cap 10, it may be sized to receive the exterior surface of the inlet conduit 8 therewithin. In this example, the cavity 56 may be configured so as to securely cap the inlet port 9. The peripheral edge of the cavity 56 does not have to be smooth and may include internal ridges or projections to further aid in closing or securely capping the inlet port 9. The cavity 56 may be replaced with a male plug constructed essentially as or close to the construction of the male plug 12 or the male plug may be positioned within the cavity 56. The flange 52 with the cavity 56 may be also adapted with the above described slot 33 of any exemplary embodiments. The exterior peripheral shape or the contour of the flange 52 may include a substantially planar surface opposite the surface having the cavity 56 therein and is not a limiting feature in this and other exemplary embodiments and can be of any shape.

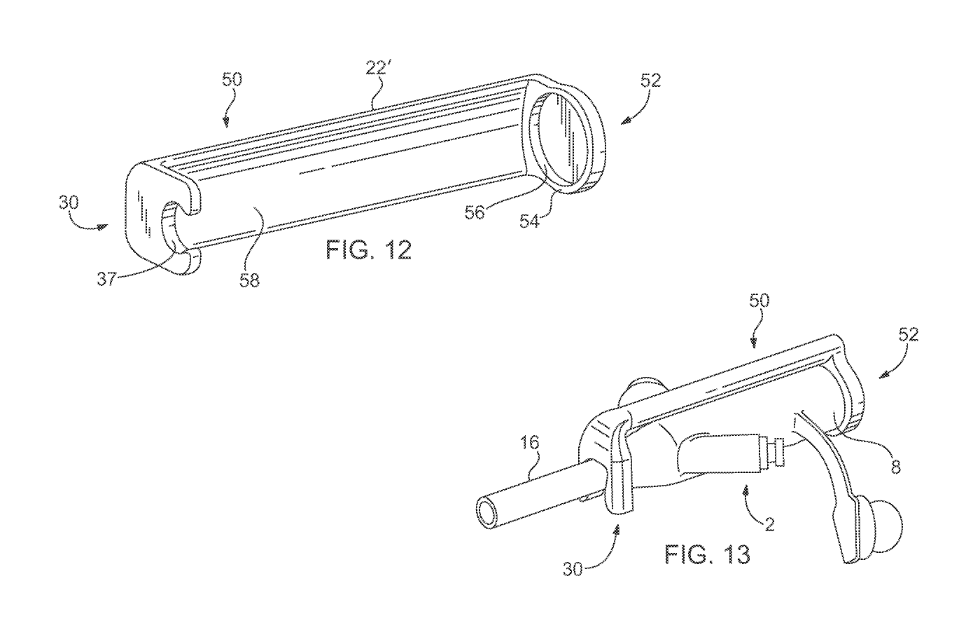

During use, the exterior surface 24 of the elongated body 22 is positioned either in a direct abutting contact or at a distance from an exterior surface of the body 4. The flange 30 is positioned during use of the device 50 at the outlet conduit 6 so that the outlet tube 16 is at least inserted into the opening 34 or 38, while the flange 52 is positioned at the inlet conduit 8 to either directly close or securely cap the inlet port 9, as is shown in FIG. 13, or abut the exterior surface of the integral movable cap 10 when its plug 12 is inserted into the inlet port 9. The device 50 may be manufactured from any one of plastic material, metallic material and a combination thereof. The device 50 may be provided as a unitary one-piece construction, for example by a molding or a casting process, or a construction wherein one or both flanges 30 and 52 are permanently, rigidly or releasably connected to the elongated body 22 in a manner described above in the exemplary embodiment of the device 20 of FIGS. 6-9. Also shown in FIG. 12 are a single cavity 37 and a concave shape of the interior surface 58 of the elongated body 22′. The exterior peripheral shape or the contour of the flange 52 of FIGS. 11-13 is not a limiting feature in this and other exemplary embodiments and can be of any shape and or thickness.

It is also contemplated in the exemplary embodiment of FIGS. 11-13 that the flange 52 can be replaced with the above described flange 30, particularly when the Y-port feeding adapter 2 employs the movable cap 10. Thus the device 20 of FIGS. 1-10 may be adapted and configured, by changing, for example, the length of the elongated portion 22, to both lock the connection between the inlet tube 18 and the inlet port 9 of the Y-port feeding adapter 2 and/or close or securely cap the inlet port 9 when the Y-port feeding adapter 2 employs the movable cap 10 having its plug 12 being inserted into the inlet port 9 when the inlet tube 18 with its fitting 19 are removed from the inlet port 9.

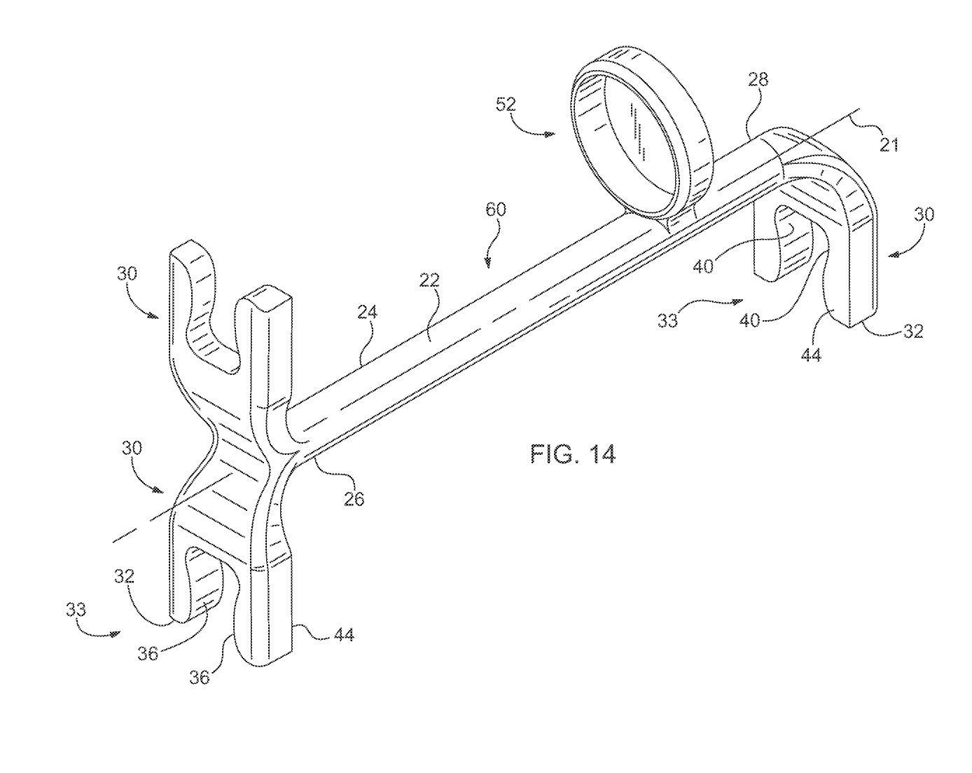

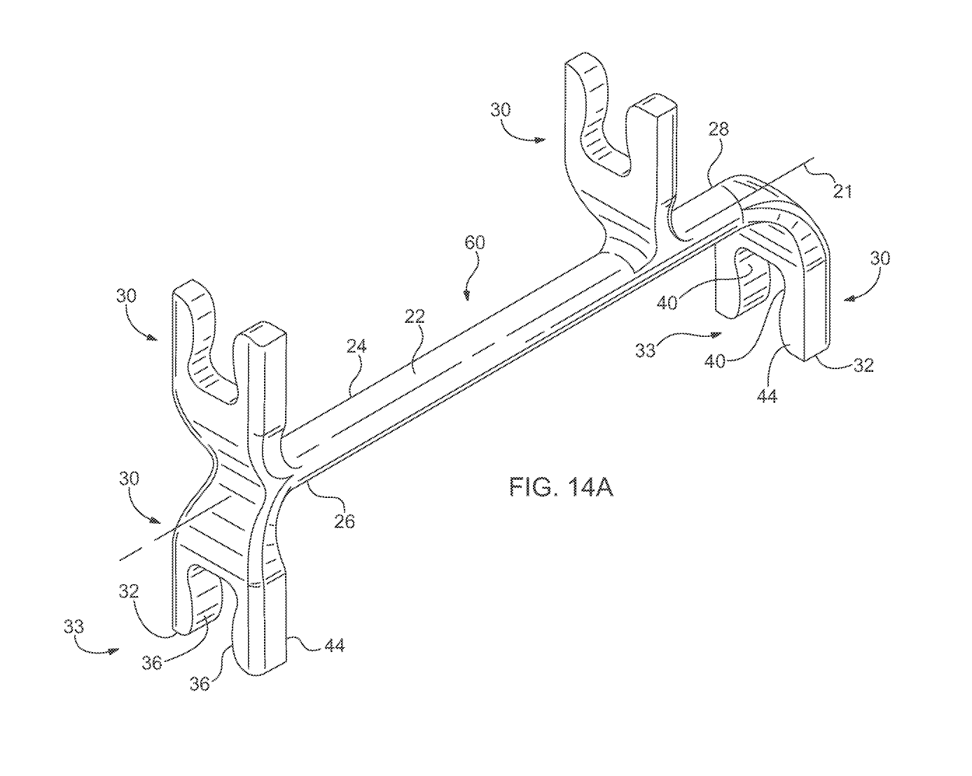

In yet another exemplary embodiment of FIG. 14, the device or the article of manufacture 60 includes the above described elongated body 22, a single flange 30 at one end of the elongated body 22 defining a first retention or locking portion, a pair of flanges 30 at an axially opposite end of the elongated body 22, referenced with a numeral 26, defining a second retention or locking portion and a flange 52, defining a third retention or locking portion, being connected, either directly or indirectly, to the exterior surface 24 of the elongated body 22 mediate the flanges 30 and extending in a direction of an upper flange 30 at the end 26. Each flange 30 has the slot 33 formed through a thickness thereof. The surface of the flange 52 being opposite the surface having the cavity 56 is contemplated in the example of FIG. 14 as a substantially planar surface, subject to manufacturing tolerances. The device 60 is either used in a manner of the above described device 20 to lock the connection between the inlet port 9 and the inlet tube 18 by employing the lower flanges 30 or in a manner of the above described device 50 to either directly or indirectly close or cap the inlet port 9, by employing the upper flange 30 at the end 26 of the elongated body 22 and the flange 52. The device 60 of the exemplary embodiment of FIG. 14 is illustrated as a unitary, one piece construction wherein the flanges 30 and 52 are permanently or rigidly fixed to the elongated body 22, although one or more flanges 30 and 52 may be releasably connected to the elongated body 22 in the manner described above in the exemplary embodiment of the device 20 of FIGS. 6-9. Furthermore, the upper flange 30 and the flange 52 do not have to extend in a direction opposite to the direction of the lower flanges 30 and may be inclined at any angle relative thereto, sufficient to provide independent use of the lower flanges 30 and the upper flange 30 and the flange 52. In other words, the direction of upper flange 30 and the flange 52 may be different than the direction of the lower flanges 30, while providing such independent use of operation. By way of one example only, the upper flange 30 and the flange 52 may be rotated ninety (90) degrees in either clockwise or counter-clockwise direction relative to the lower flanges 30.

Now in a reference to FIG. 14A, it is also contemplated that the flange 52 can be replaced with the above described flange 30, particularly when the Y-port feeding adapter 2 employs the movable cap 10. Thus, the locking device 60 in the example of FIG. 14 is illustrated as including four (4) identical flanges 30.

The elongated body 22 is illustrated in FIGS. 14-14A as having a uniform round cross-sectional shape in the plane normal to the length of the elongated body 22. The front and rear views are illustrated in FIGS. 14-14A as being identical to each other.

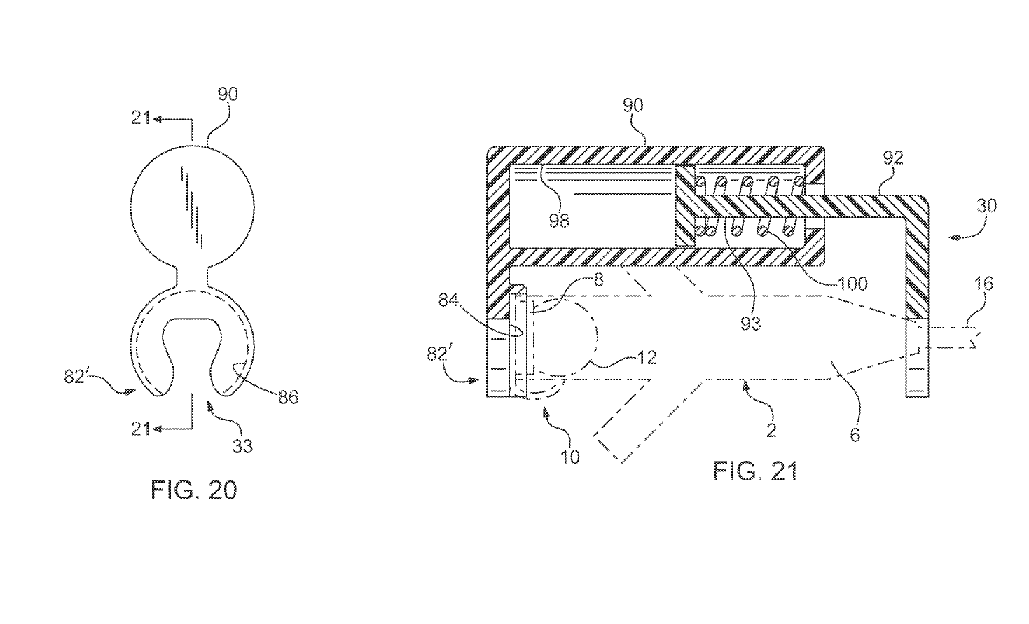

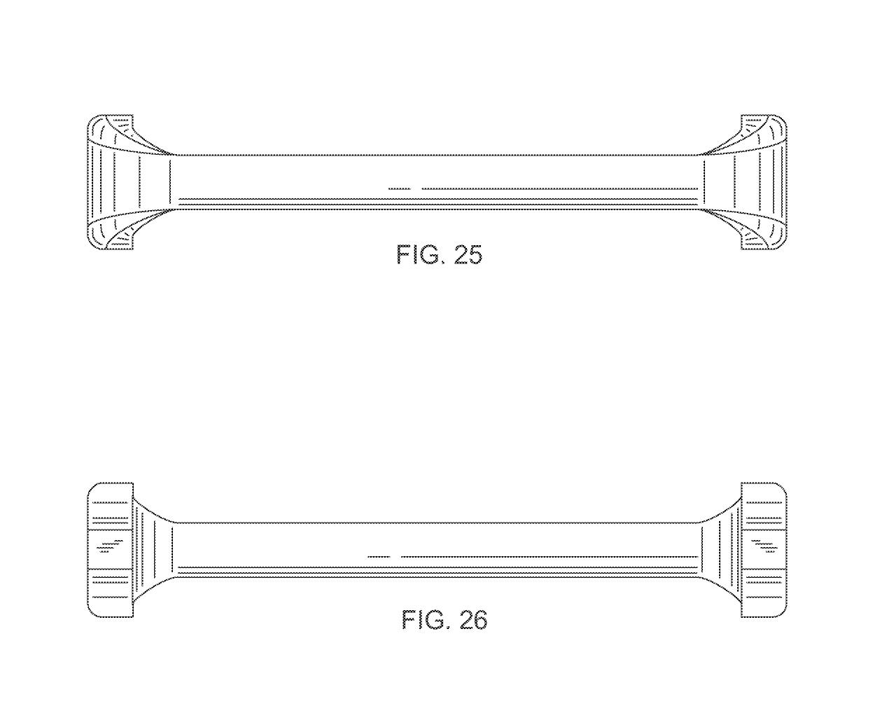

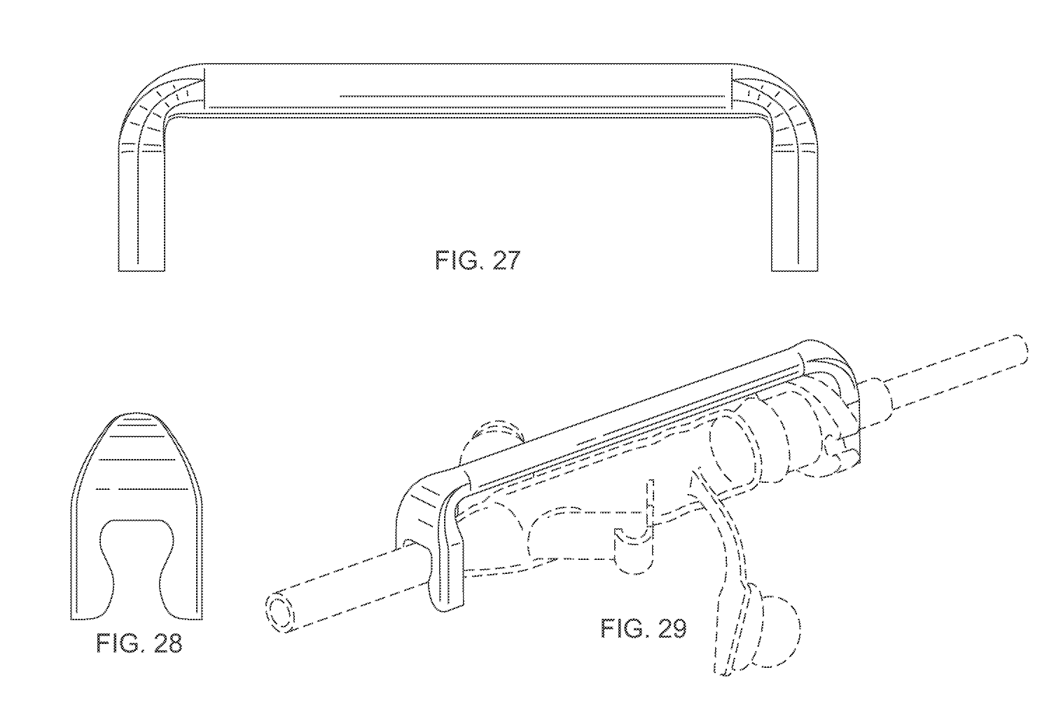

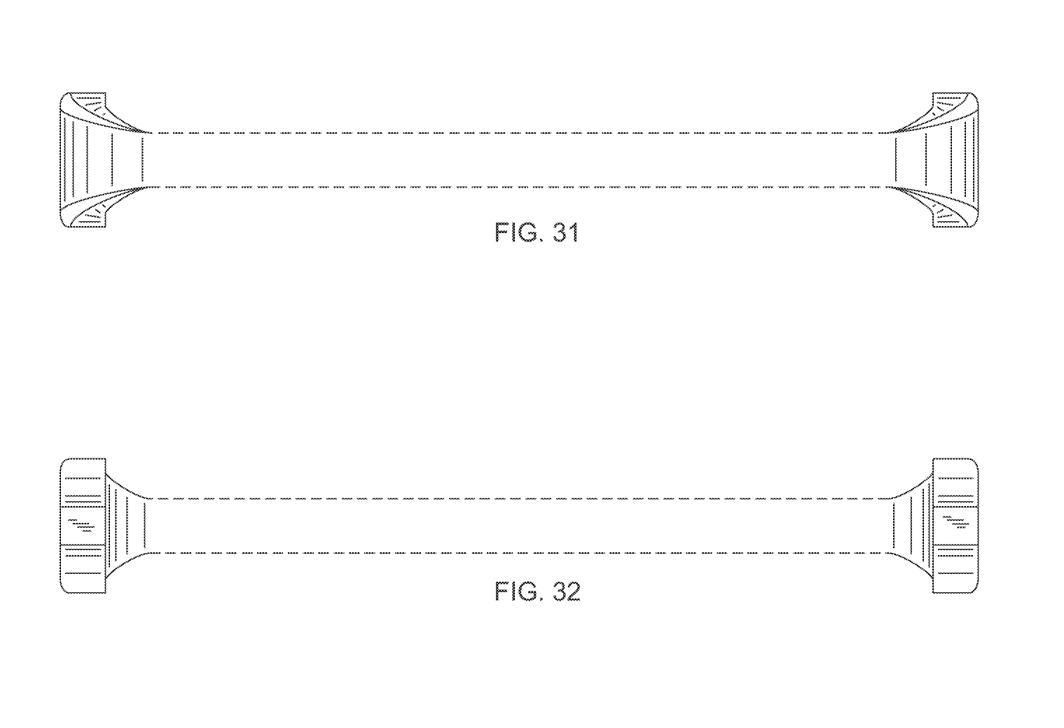

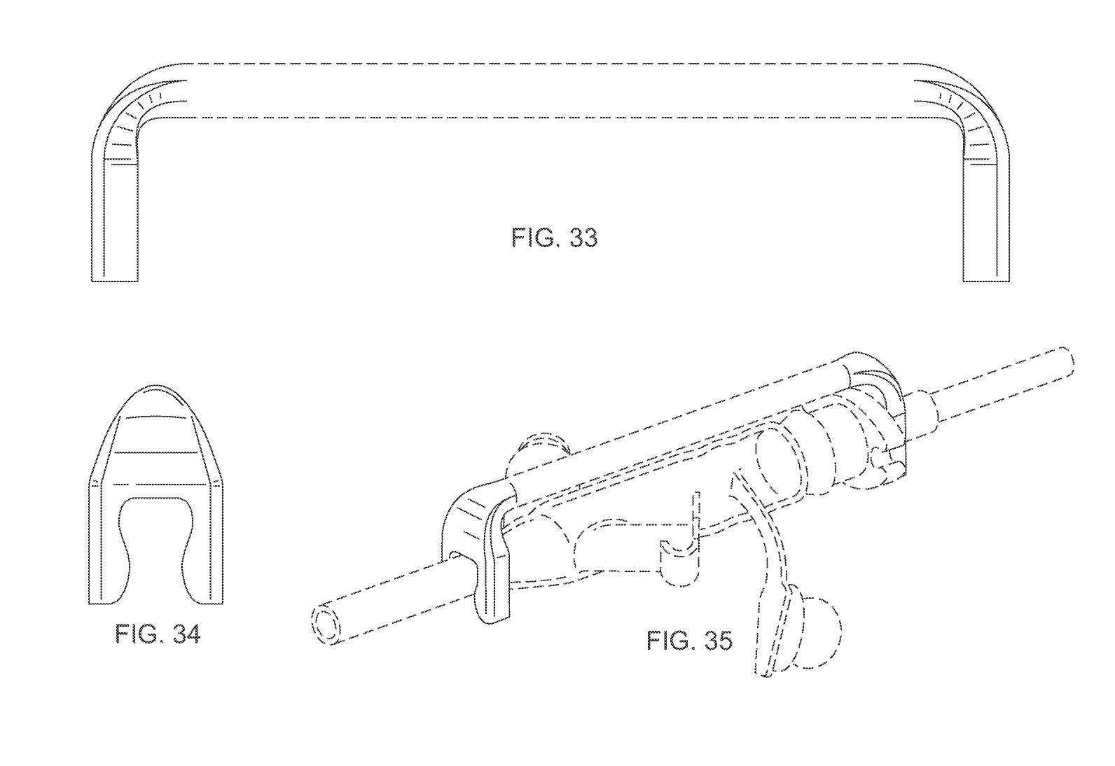

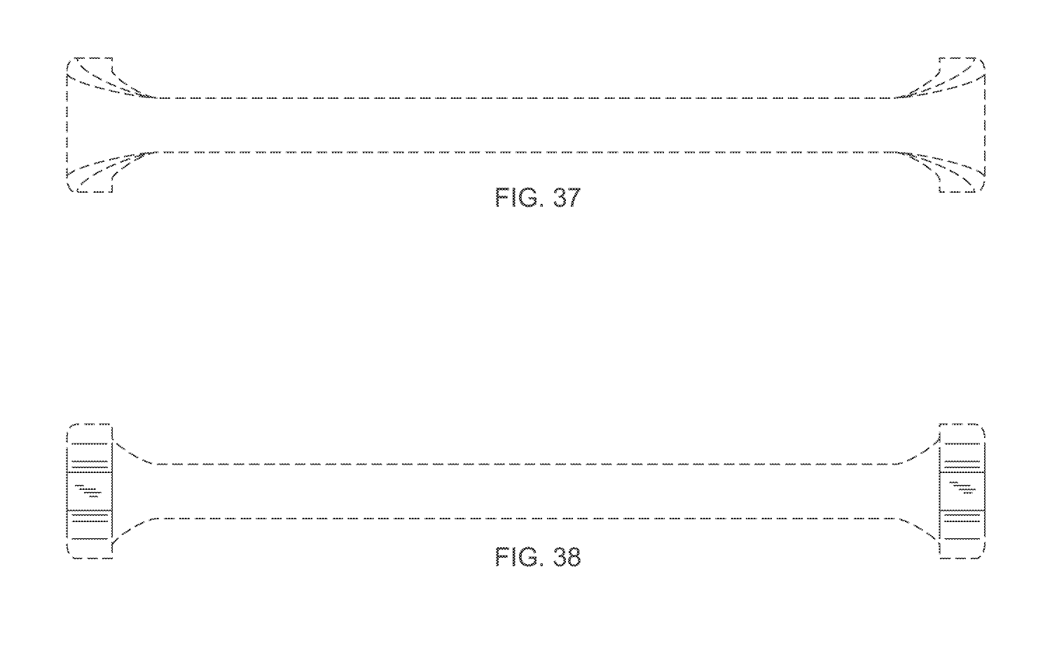

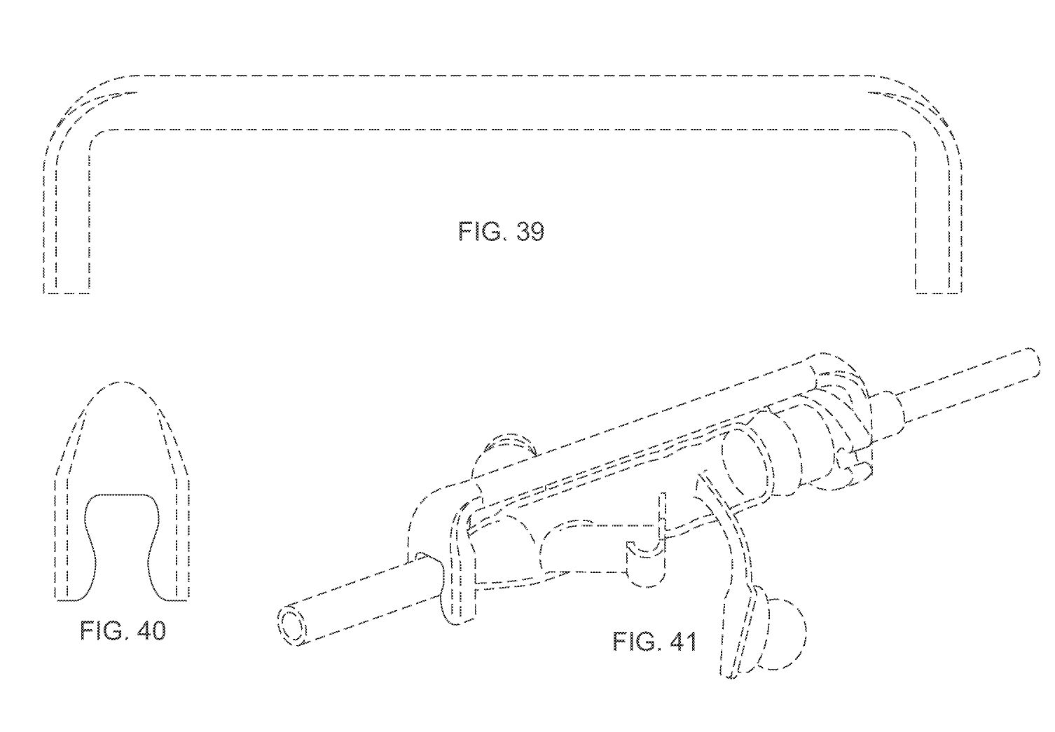

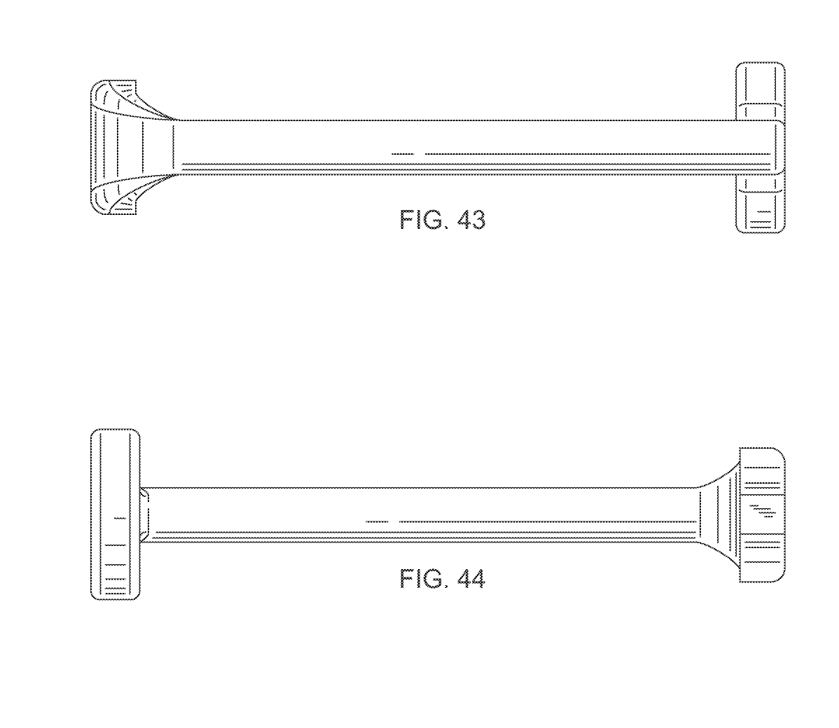

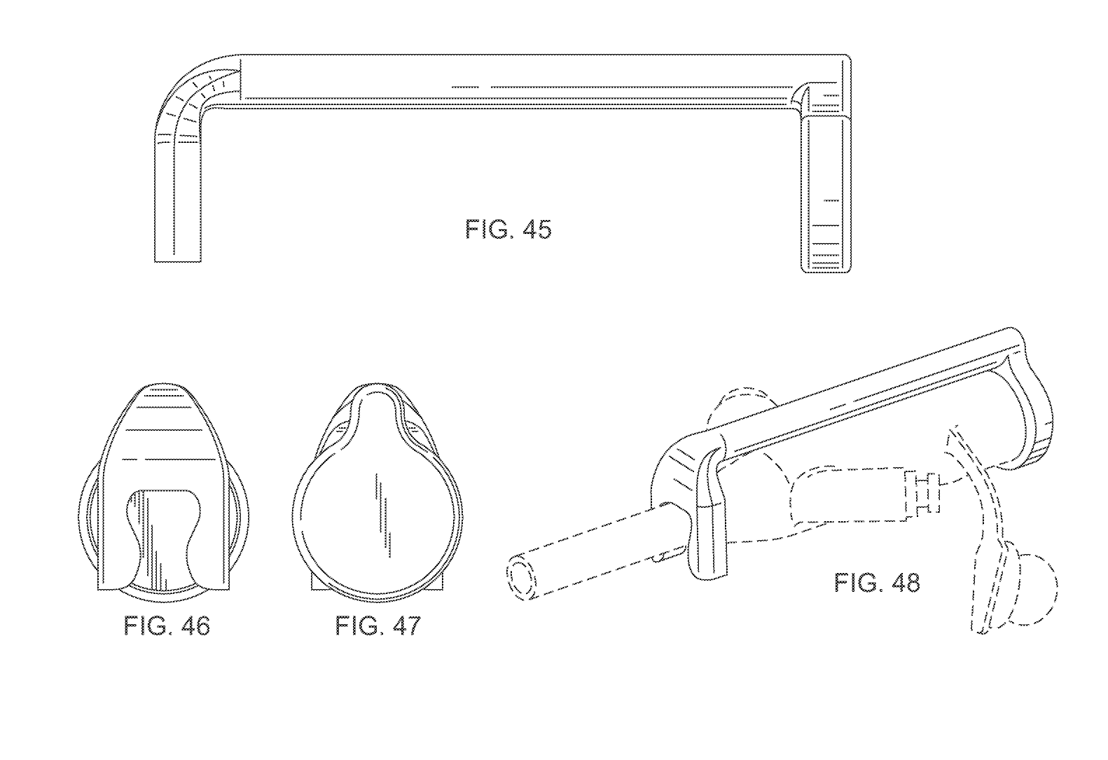

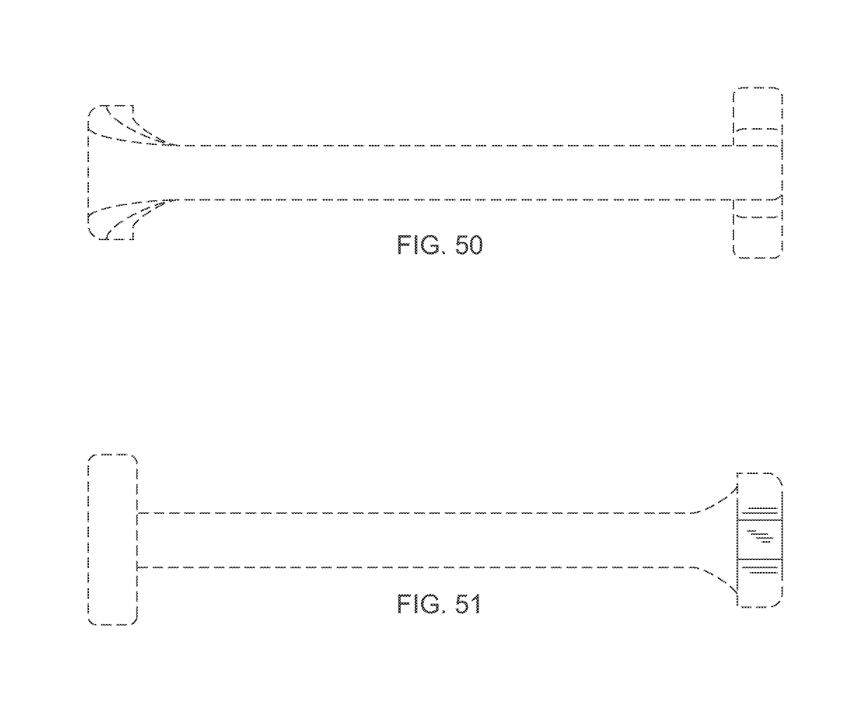

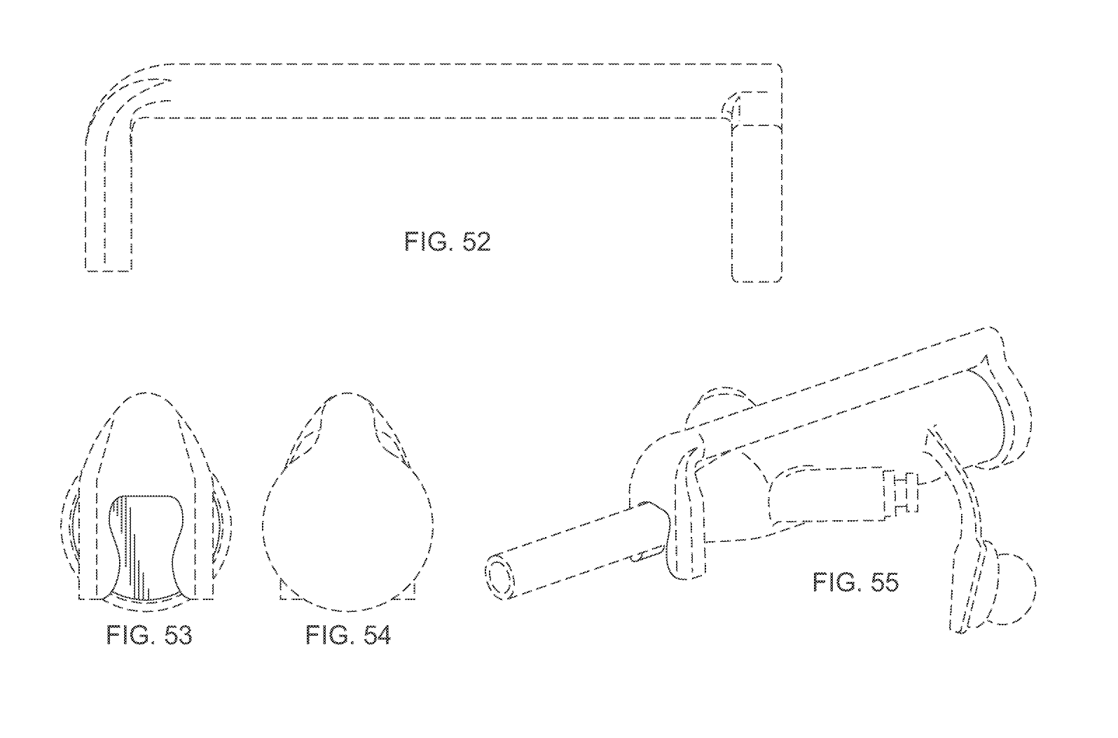

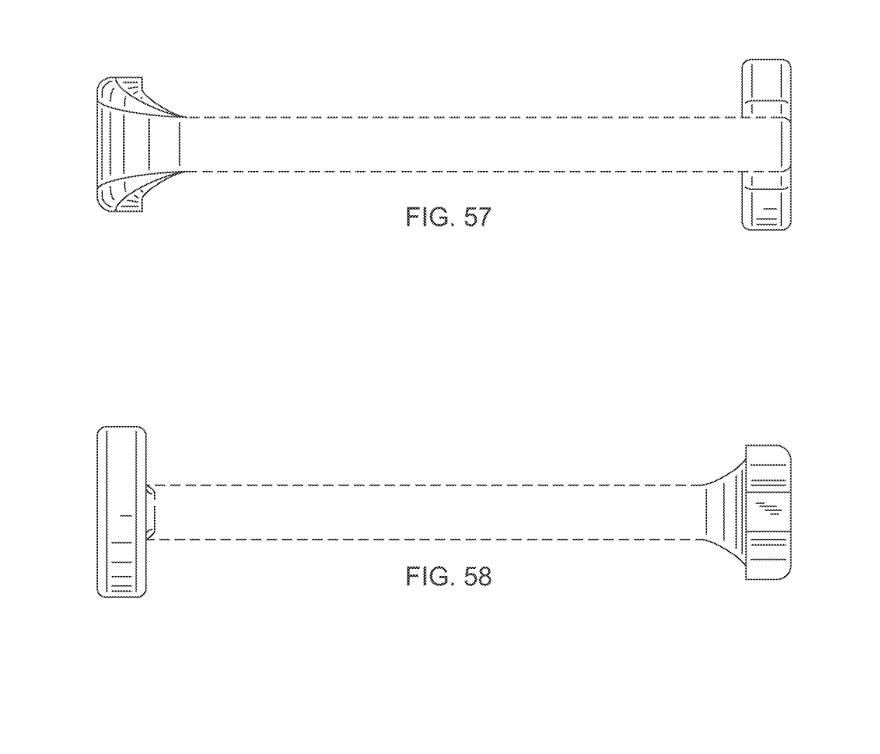

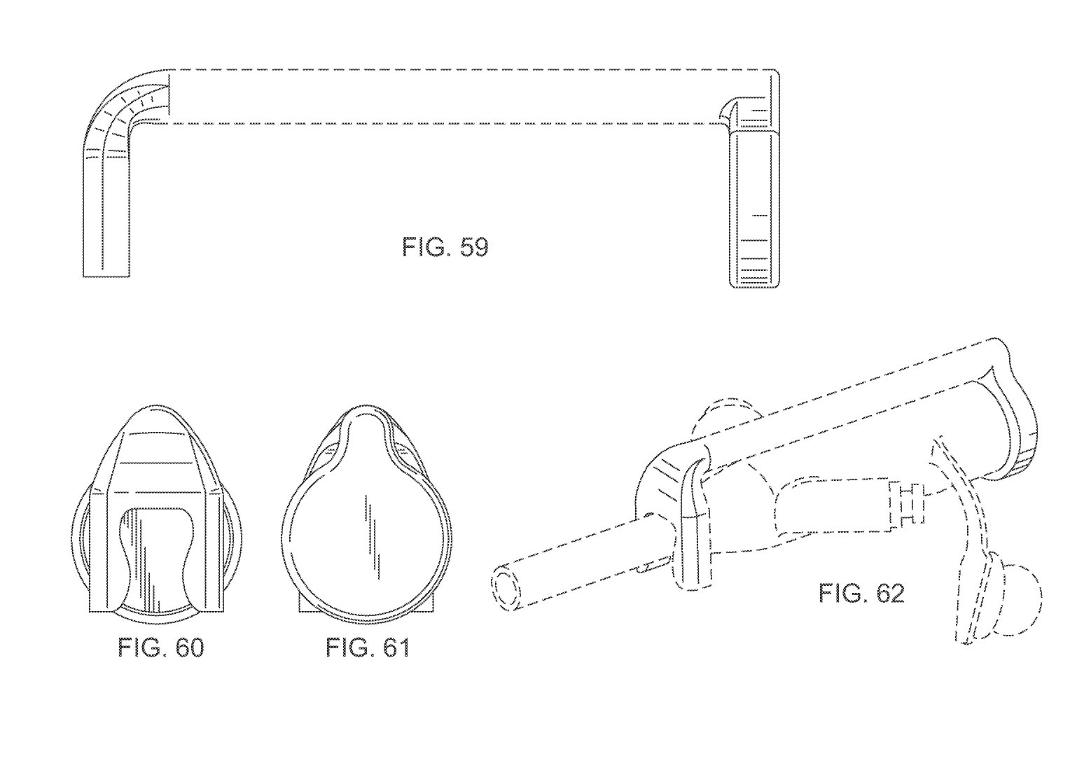

FIGS. 14 and 14A also illustrate (when presented without reference numerals and further in a conjunction with the relevant Brief Description of the Drawings and in a reference to FIGS. 20 and 24-62) an ornamental design for a locking device for a medical tube coupling. The claimed locking device is configured and operable to lock a connection between an inlet tube and an inlet port of the medical tube coupling or close or cap the inlet port of the medical tube coupling.

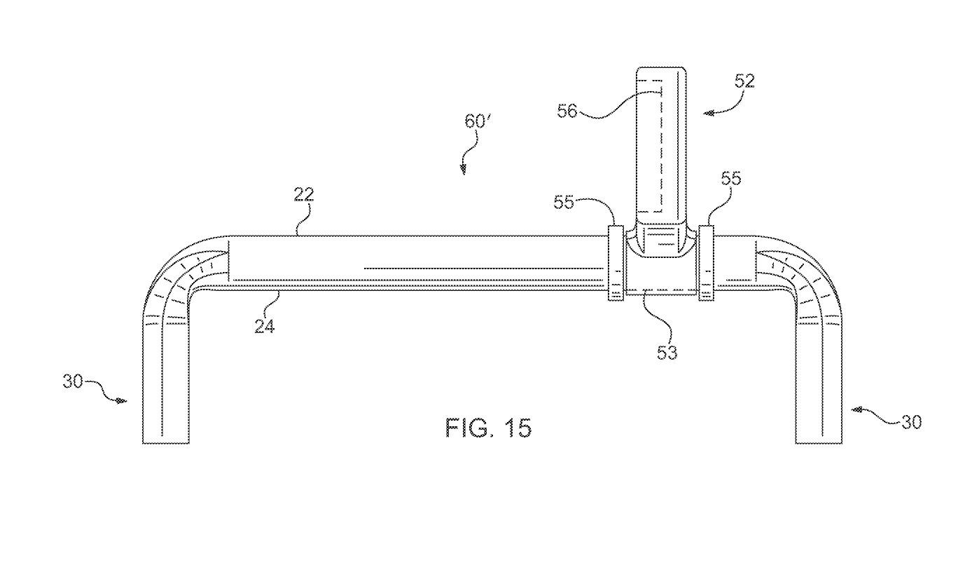

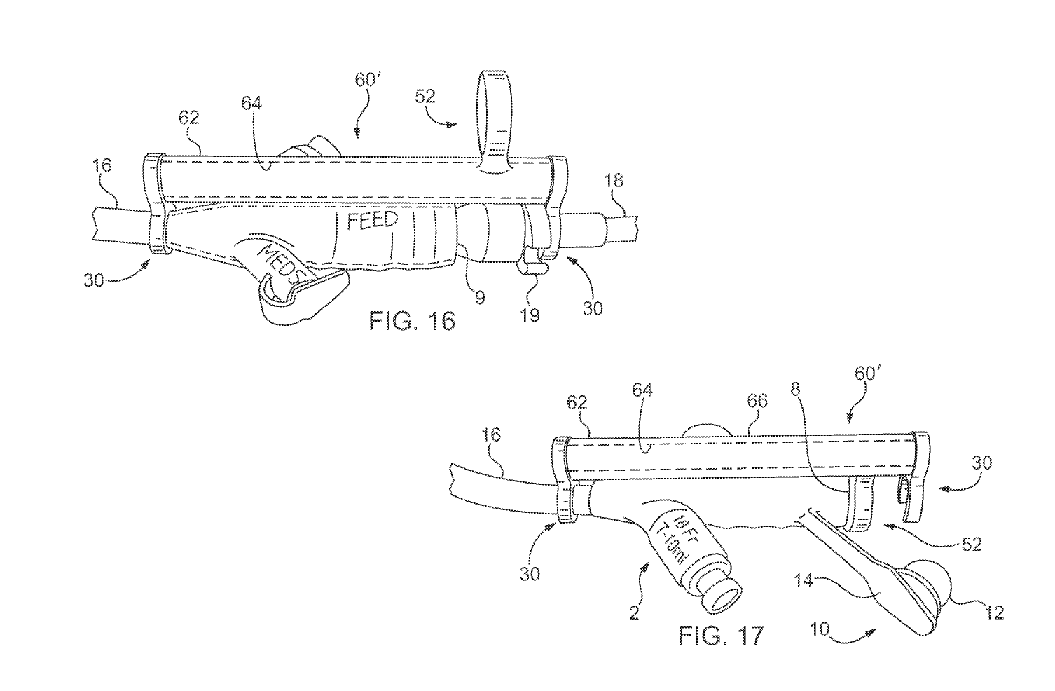

In a further exemplary embodiment of FIGS. 15-17, the device or the article of manufacture 60′ provides for a rotational or a pivotal connection of one or both flanges 30 and/or the flange 52 about the length of the elongated body 22. In one example of FIG. 15, the flange 52 is adapted with an aperture 53 sized to receive the exterior surface 24 of the elongated body 22 therethrough. The exterior surface 24 can be also adapted with a pair of spaced apart projections 55 caging the thickness of the flange 52 and preventing a movement thereof along the length of the elongated body 22 while permitting a rotational or a pivotal movement of the flange 52 about the longitudinal axis 21. Each projection 55 may be of a continuous type on the exterior surface 24 of the elongated body 22 or may be provided as a single tab. In another example of FIGS. 16-17, the device 60′ may be adapted with a sleeve 62 having an interior surface 64 thereof sized to receive the exterior surface 24 of the elongated body 22 so that the sleeve 62 is free to rotate or pivot thereon. The flange 52 is then directly or indirectly attached to the exterior surface 66 of the sleeve 62 for a rotation or pivoting therewith. The flange 52 in this exemplary embodiment does not have to rotate one hundred and eighty (180) degrees relative to the flanges 30 and may pivot or rotate sufficiently to provide independent operation either in a manner of the above described device 20 or in a manner of the above described device 50. As it was described before, the cavity 56 may be omitted when the Y-port feeding adapter 2 is provided without the movable cap 10. FIG. 15 also illustrates a front or rear view of the device of FIG. 1, when the flange 52 and the projections 55 are removed.

In a further example, the device 60′ may include the flange 52 being fixed, in a non-rotational manner, to the exterior surface 24 of the elongated body 22 and wherein the flanges 30 are connected to respective ends 26 and 28 in a rotational or a pivotal manner. For example, the above described stems 46, 46′ and bores 48, 48′ or fastener 49 may be sized to provide a rotational or a pivotal connection without unintended separation of the flange(s) 30 from the elongated body 22 during use of the device 20.

In another exemplary embodiment, the device or an article of manufacture may be provided as an assembly of two or more components to lock the connection between the inlet or feeding tube 18 and the inlet port 9 of the Y-port feeding adapter 2 and/or close or securely cap the feeding tube inlet port 9 of the Y-port feeding adapter 2, wherein the operable length of the device is adjustable by the user.

The device 60′ of FIG. 16 is operable to lock the connection between the feeding tube 18 and the inlet port 9, while the device 60′ of FIG. 17 is operable to directly abut and securely cap the inlet port 9 of the Y-port feeding adapter 2 when the feeding tube 18 is removed from the inlet port 9.

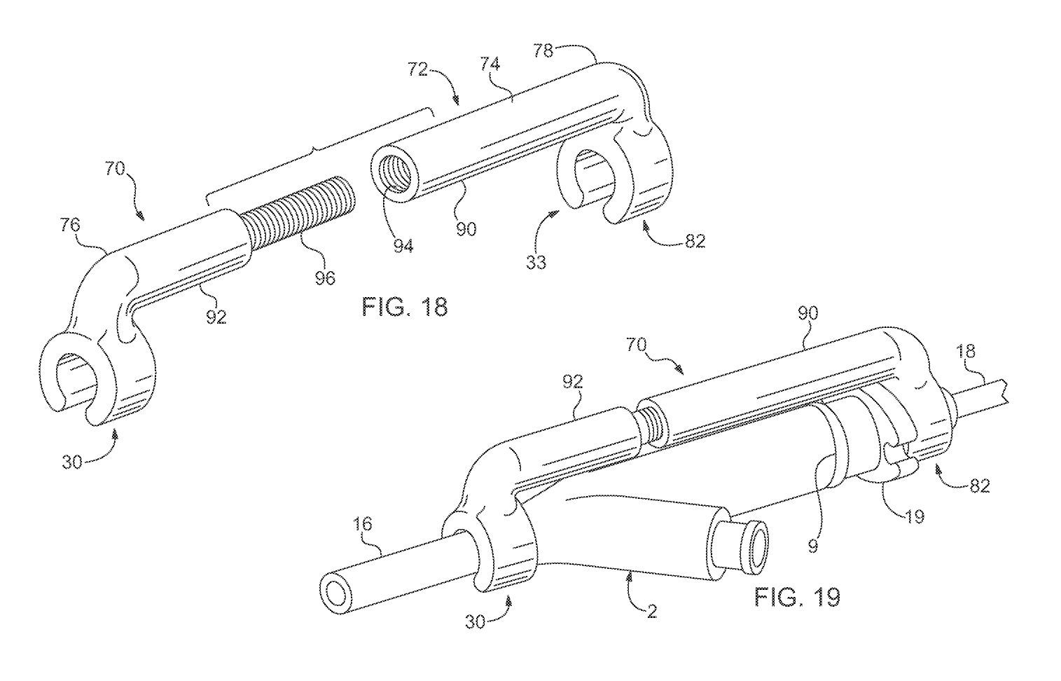

In one example of FIGS. 18-19, the device or the article of manufacture 70 comprises an elongated portion 72 with an exterior surface 74 and a pair of a longitudinally opposite ends 76 and 78. A flange 30, constructed and attached, in accordance with any one of the above described examples, to one end of the elongated portion 72, referenced by a numeral 76. A flange 82 is attached, by any one of the above described examples, to the opposite end of the elongated portion 72, referenced by a numeral 78. The flange 82 may be constructed essentially identical to the above described flange 30 and may also include the above described slot 33, in accordance with any one of the above described examples, when the Y-port feeding adapter 2 is provided with the movable cap 10. The flange 82 may be also constructed essentially identical to the above described flange 50 and may include the cavity 56, particularly when the Y-port feeding adapter 2 is provided without the movable cap 10.

The exterior surface 74 does not have to be smooth and/or does not have to have a round peripheral shape and may be provided with any gripping features being, without limitations projections, ridges, surface roughness, coating, elastomeric sleeve, etc and/or other peripheral shapes having, by way of one example only, one or more flat surface portions. By way of one example only, the exterior surface 74 may define a hexagon cross-sectional shape.

In operation, the device 70 is selectively operable in a first position to lock the inlet tube 18 being inserted into the inlet port 9 or in the second position to close or securely cap the inlet port 9 either with or without the movable cap 10 in place. Accordingly, the length of the elongated portion 72 is made as user adjustable during use of the device 70 and includes two body portions 90 and 92 that are connected for a longitudinal movement relative to one another.

One of the body portions, for example the body portion 90, may be provided with an internal or female thread 94 and the other body portion 92 may be provided with a complementary external or male thread 96.

In another example of FIGS. 20-21, one of the body portions, for example the body portion 90, is provided with a bore 98 and at least an end 93 of the other body portion 92 is sized to be received within the bore 98. Furthermore, a spring 100 is fixed within the bore 98 so that the body portions 90 and 92 and, more particularly, the flanges 30 and 82′ move or are biased toward each other during use, thus allowing the device 70 to be used either in the first or the second position. Although the spring 100 is illustrated in FIG. 21 as a coiled spring, it may be manufactured from an elastomeric material being compressible and resilient sufficiently to move or bias the flanges 30 and 82′ toward each other.

As is further shown in FIG. 20, the surface 84 of the cavity 86 is positioned during use to abut the exterior surface of the strap 14 when the plug 12 is inserted into the inlet port 9, while the cavity 86 is sized to receive the terminal portion of the strap 14. The slot 33 in the flange 30 and/or flange 82 can be of any shape in a further accordance with FIGS. 3-5.

The above described threads 94, 96 and the spring 100 in FIGS. 18-21 provide means for moving or biasing the first and second flanges, 30, 82 and 82′ respectively toward or apart from each other and may further provide means for moving or biasing the first and second flanges, 30, 82 and 82′ respectively toward or apart from each other without use of any tools.

It is also contemplated that the locking device of any of the above described exemplary embodiments may be permanently attached to the Y-port feeding adapter 2, for example to the exterior surface of the outlet conduit 6. Term “permanently” is intended to mean that the slot 33 of the flange 30 may be made integral with the Y-port feeding adapter 2. In other words, the exterior surface portion of the Y-port feeding adapter 2 and/or the exterior surface portion of the tube 16 or 18 may define the contour of the slot 33. Term “permanently” is also intended to mean that the inner surfaces of the slot 33 may be attached to exterior surface portion of the Y-port feeding adapter 2 by way of adhesives (not shown). Thus, the invention also provides a novel medical tube coupling having a locking device integrated thereinto, configured and operable to lock a connection between an inlet tube and an inlet port of the medical tube coupling or close or securely cap the inlet port of the medical tube coupling.

The method of using any one of the above exemplary devices for locking a connection between an inlet tube and an inlet port of a medical tube coupling and/or closing or securely capping a an inlet port of the medical tube coupling comprises the step of positioning the elongated body of the device in one of a close proximity, adjacent and in an abutting engagement with the body of the medical tube coupling, the step of attaching a first flange at the outlet end of the medical tube coupling by inserting the outlet tube into the slot and the step of either locking, using a second flange, the fitting of the inlet tube being inserted into the inlet port, wherein the inlet tube is received within a slot in the second flange, or closing or securely capping the inlet port with the other flange when the fitting of the inlet tube is removed from the inlet port. The method contemplates that either one of the provided flanges may be selected as a first flange. In other words, the flange positioned at the inlet end of the medical tube coupling may be the first flange being attached thereto. The method may include the optional step of adjusting the length of the device and/or the optional step of rotating one or more flanges. The method may also include the optional step of assembling or attaching one or more flanges to the elongated body of the device in accordance with the examples of FIGS. 6-9. The method may further include the optional step of using a spring or threads to adjust the length of the device or, more specially, adjust a distance between inner surfaces of the opposing flanges in a non-ratcheting manner and without use of any tools. The method contemplates the step of providing the medical tube coupling with or without the integral movable cap at the inlet end thereof, particularly when the device is adapted for locking a connection between a catheter and a male fitting used in administering intravenous (IV) fluids to a patient. The method may include the step of adapting the device with three flanges and using these three flanges to either lock the connection between the inlet tube and the inlet port of the medical tube coupling or close or securely cap the inlet port of the medical tube coupling when the inlet tube is removed therefrom. When three flanges are provided, the method may include an optional step of rotating an inner flange positioned mediate the two outer flanges. The method may also include the step of providing the device with four flanges and using these four flanges, in two independent pairs, to either lock the connection between the inlet or feeding tube and the inlet port of the medical tube coupling or close or securely cap the inlet port of the medical tube coupling when the inlet tube is removed therefrom.

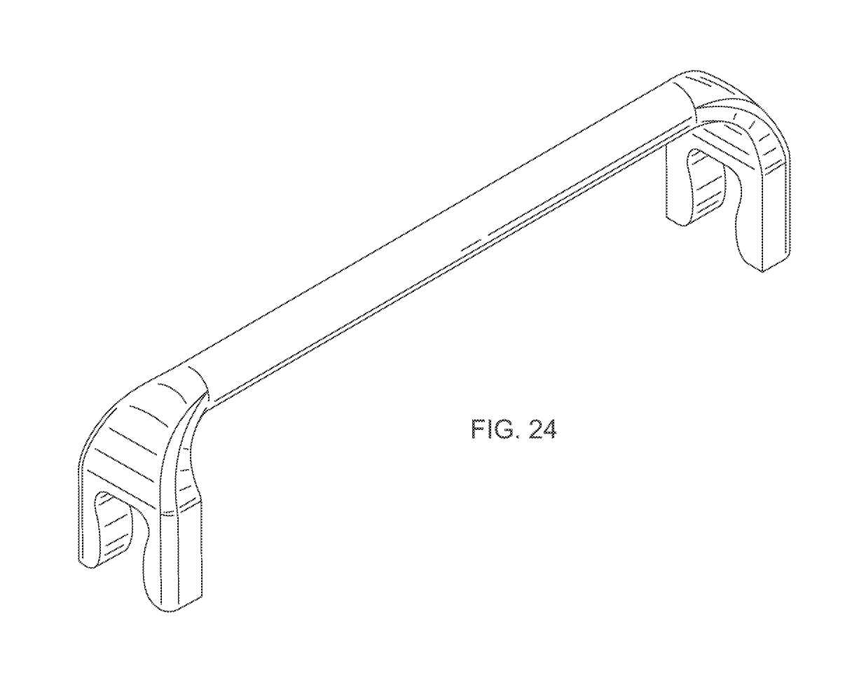

FIGS. 24-29 illustrate an ornamental design for one exemplary locking device for a medical tube coupling. The claimed locking device is configured and operable to lock a connection between an inlet tube and an inlet port of the medical tube coupling or close or securely cap the inlet port of the medical tube coupling, wherein the medical tube coupling is illustrated for environmental purposes as a Y-port feeding adapter.

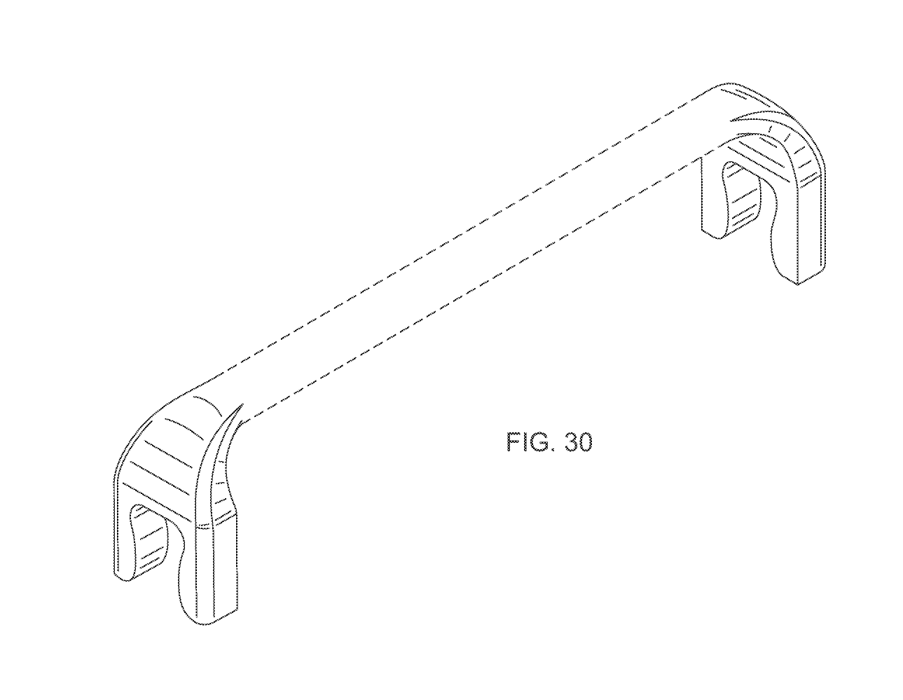

FIGS. 30-35 illustrate an ornamental design for another exemplary locking device for a medical tube coupling. The claimed locking device is configured and operable to lock a connection between an inlet tube and an inlet port of a medical tube coupling or close or securely cap the inlet port of the medical tube coupling, wherein the medical tube coupling is illustrated for environmental purposes as a Y-port feeding adapter.

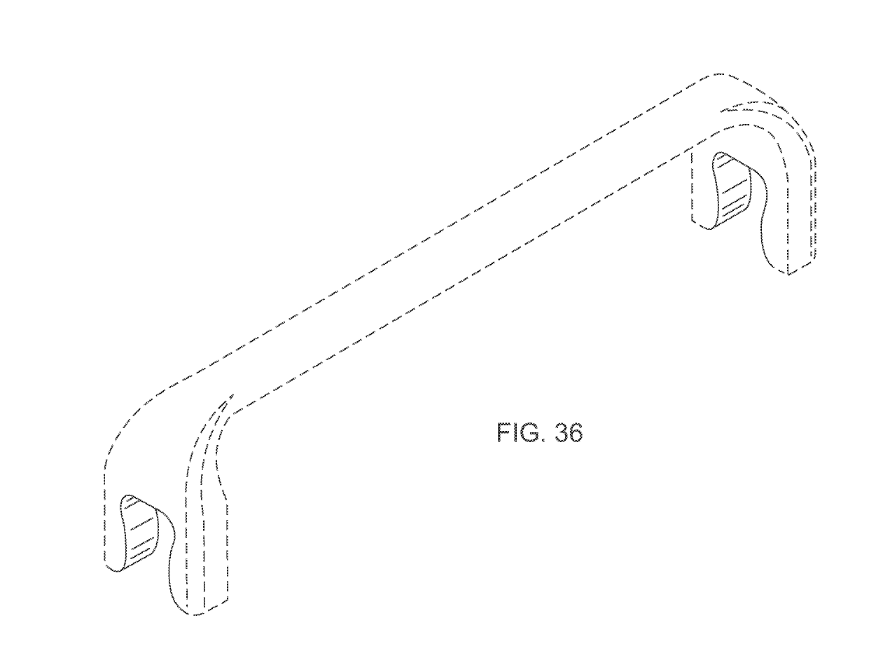

FIGS. 36-41 illustrate an ornamental design for another exemplary locking device for a medical tube coupling. The claimed locking device is configured and operable to lock a connection between an inlet tube and an inlet port of the medical tube coupling or close or securely cap the inlet port of the medical tube coupling, wherein the medical tube coupling is illustrated for environmental purposes as a Y-port feeding adapter.

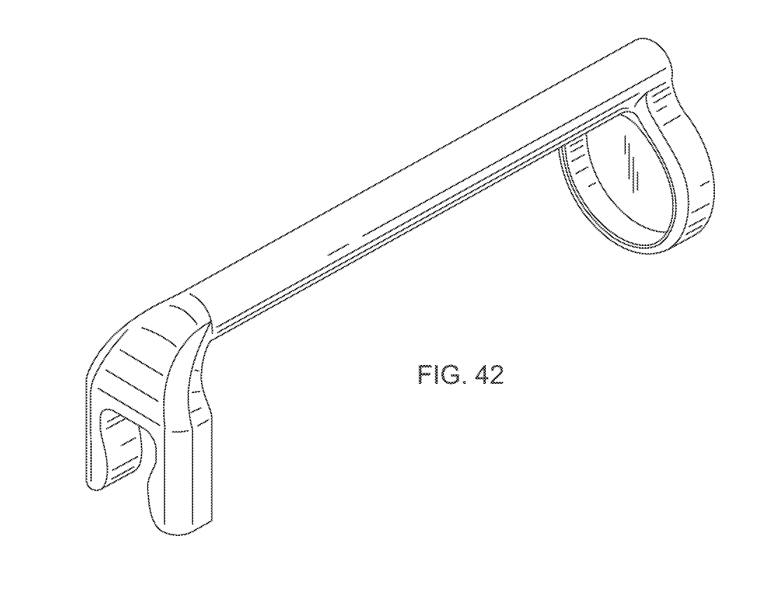

FIGS. 42-48 illustrate an ornamental design for another exemplary locking device for a medical tube coupling. The claimed locking device is configured and operable to close or securely cap an inlet port of the medical tube coupling, illustrated for environmental purposes as a Y-port feeding adapter.

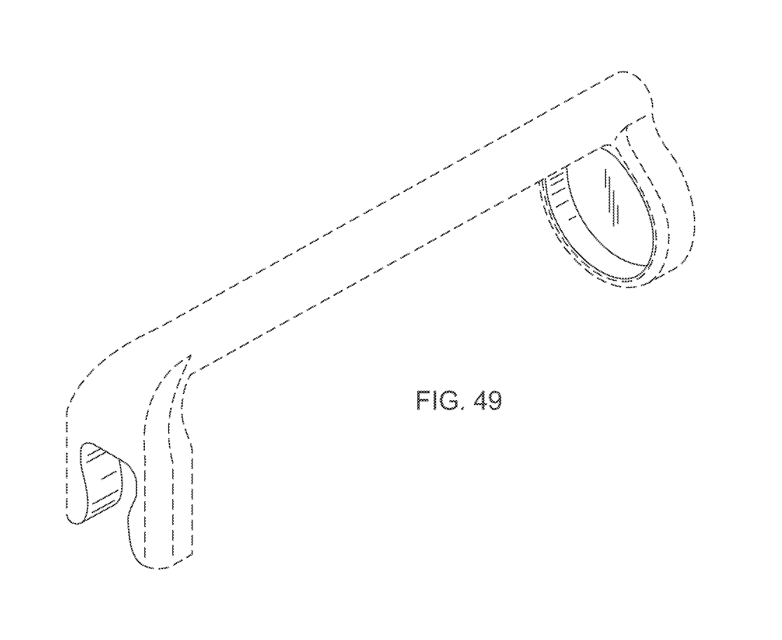

FIGS. 49-55 illustrate an ornamental design for another exemplary locking device for a medical tube coupling. The claimed locking device is configured and operable to close or securely cap an inlet port of the medical tube coupling, illustrated for environmental purposes as a Y-port feeding adapter.

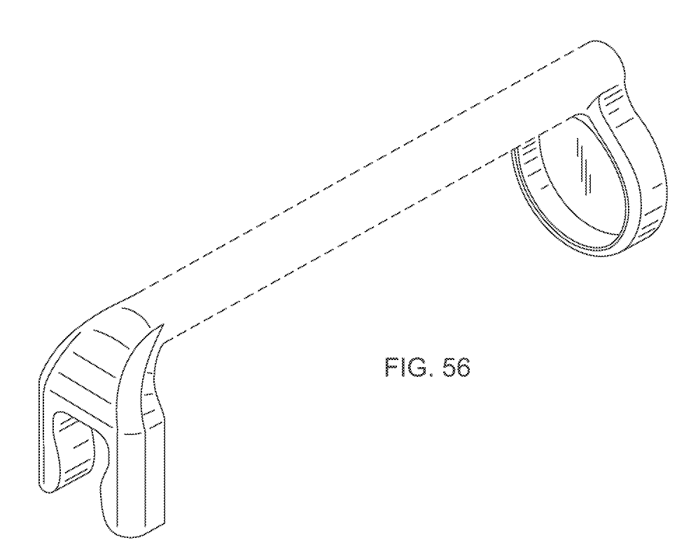

FIGS. 56-62 illustrate an ornamental design for another exemplary locking a device for a medical tube coupling. The claimed locking device is configured and operable to close or securely cap an inlet port of the medical tube coupling, illustrated for environmental purposes as a Y-port feeding adapter.

Although illustrated in a combination with the Y-port feeding adapter for enteral feeding, it would be apparent to a skilled worker that the invention may be also adapted to lock a connection between a female catheter and a male fitting used in administering IV fluids to a patient. By way of one example, the above described exemplary embodiments can be adapted for the connection between a female catheter and a male fitting as taught in U.S. Pat. No. 4,997,421 issued to Palsrok et al. on Mar. 5, 1991, wherein one flange 30 is configured to be positioned near a sheath end of the female catheter and receive the sheath through the slot 33 and the opposite flange is configured to receive the IV tube and/or close or securely cap the inlet aperture when the male connector is removed. By way of another example, the above described exemplary embodiments can be adapted for the connection between a female catheter and a male fitting as taught in U.S. Pat. No. 4,826,486 issued to Palsrok et al. on May 2, 1989. By way of yet another example, the above described exemplary embodiments can be adapted for the connection between a female catheter and a male fitting as taught in U.S. Pat. No. 4,230,109 issued to Geiss on Oct. 28, 1980.

The invention may be also adapted to lock a connection or a connecting member between breathing or respiratory tubing members and/or connection or a connecting member between a urinary tubing and a drainage bag.

In one exemplary embodiment, the flange 30 at the outlet end of the medical tube coupling may be positioned and operable to wedge, in a releaseable manner, the outlet tube within the slot 33 so as to apply pressure thereonto, where the pressure may result in a slight flattening of the outlet tube 16 but not sufficient to impact a passage of the fluid or feed therethrough. When the locking device is configured and/or operable to securely lock a connection between the medical tube coupling and the medical tube, the spacing between flanges 30 is so pre-selected or adjusted that the opposite flange 30 at the inlet end 8 is positioned to directly abut the flange or a surface portion of the male fitting on the inlet tube 18, thus applying force onto the medical tube coupling along the longitudinal axis 21. When the locking device is configured and/or operable to securely cap the inlet end 9 of the medical tube coupling, the spacing between flange 30 and flange 52 is so pre-selected or adjusted that the flange 52 is positioned to apply force directly onto the inlet end 8 or the plug 12, plugging the inlet port 9, along the longitudinal axis 21.

In another exemplary embodiment, when the locking device is configured to securely lock a connection between the medical tube coupling and the medical tube, the flange 30 at the outlet end of the medical tube coupling is positioned and operable to wedge, in a releaseable manner, the outlet tube within the slot 33 so as to apply pressure thereonto, where the pressure may result in a slight flattening of the outlet tube 16 but not sufficient to impact a passage of the fluid or feed therethrough. The spacing between flanges 30 is so pre-selected or adjusted that the inner surface of the flange 30 at the inlet end 8 is spaced from the flange or a surface portion of the male fitting 19 and its respective slot 33 is configured to wedge the inlet tube 18 therewithin, thus preventing unintentional disengagement of the male fitting 19 from the inlet port 9 sufficient to cause leakage therefrom.

In yet another exemplary embodiment, when the locking device is configured and/or operable to securely lock a connection between the medical tube coupling and the medical tube, both flanges 30 are positioned and operable to wedge, in a releaseable manner, the respective tube within the slot 33 so as to apply pressure thereonto, where the pressure may result in a slight flattening of the respective tube but not sufficient to impact a passage of the fluid or feed therethrough. The spacing between flanges 30 may be so pre-selected or adjusted that the flange 30 at the inlet end of the medical tube coupling does not directly abut the flange or a surface portion of the male fitting on the inlet tube 18.

In a further exemplary embodiment, the flange 30 at the outlet end of the medical tube coupling may be positioned and operable for a line contact with the exterior surface of the outlet end of the medical tube coupling. When the locking device is configured and/or operable to securely lock a connection between the medical tube coupling and the medical tube, the spacing between flanges 30 is so selected that the opposite flange 30 is positioned to directly abut the flange or a surface portion of the male fitting on the inlet tube 18, thus applying force onto the medical tube coupling along the longitudinal axis 21. The outlet tube 16 and/or inlet tube 18 may or may not be wedged within the slot 33. When the locking device is configured and/or operable to securely cap the inlet end 9 of the medical tube coupling, the spacing between the flange 30 and the flange 52 is so pre-selected or adjusted that the flange 52 is positioned to apply force directly onto the inlet end 8 or the plug 12, plugging the inlet port 9, along the longitudinal axis 21.

In another exemplary embodiment, when the locking device is configured and/or operable to securely lock a connection between the medical tube coupling and the medical tube, the flange 30 at the outlet end of the medical tube coupling may be positioned and operable for a line contact with the exterior surface of the outlet end of the medical tube coupling. The spacing between flanges 30 is so pre-selected or adjusted that the inner surface of the flange 30 at the inlet end 8 is spaced apart (a distance) from the flange or a surface portion of the male fitting 19 and its respective slot 33 is configured to wedge the inlet tube 18 therewithin, thus preventing unintentional disengagement of the male fitting 19 from the inlet port 9 sufficient to cause leakage therefrom.

In a further exemplary embodiment, the flange 30 at the outlet end of the medical tube coupling may be positioned and operable to clip, in a releaseable manner, the outlet tube within the slot 33 without applying pressure thereonto, where the pressure may result in a slight flattening of the outlet tube 16. When the locking device is configured and/or operable to securely lock a connection between the medical tube coupling and the medical tube, the spacing between flanges 30 is so pre-selected or adjusted that the opposite flange 30 at the inlet end 8 is positioned to either directly abut the flange or a surface portion of the male fitting on the inlet tube 18 thus applying force onto the coupling along the longitudinal axis 21 or be positioned at a distance from the flange or the surface portion of the male fitting in a manner preventing unintentional disengagement of the male fitting from the inlet port. When the locking device is configured and/or operable to securely cap the inlet end 9 of the medical tube coupling, the spacing between flange 30 and flange 52 is so pre-selected or adjusted selected that the flange 52 is positioned to apply force directly onto the inlet end 8 or the plug 12 along the longitudinal axis 21 or be positioned at a distance from the plug 12 in a manner preventing unintentional disengagement of the plug 12 from the inlet port sufficient to cause leakage therefrom.

In another exemplary embodiment, the slot 33 in the flange 30 positioned at the outlet end of the medical tube coupling may be sized and shaped to clamp the outlet tube 16, received therewithin, in a manner that provides for a contact between an exterior surface of the outlet tube 16 and an inner surface of the slot 33, while preventing flattening of the outlet tube 16 and further in a manner that allows intended release of the flange 30 therefrom. When the locking device is configured and/or operable to securely lock a connection between the medical tube coupling and the medical tube, the spacing between flanges 30 is so pre-selected or adjusted that the opposite flange 30 at the inlet end 8 is positioned to either directly abut the flange or a surface portion of the male fitting on the inlet tube 18, thus applying force onto the coupling along the longitudinal axis 21 or be spaced apart (a distance) from such flange or a surface portion of the male fitting with its respective slot 33 either wedging or clamping an exterior surface of the inlet tube 18. When the locking device is configured and/or operable to securely cap the inlet end 9 of the medical tube coupling, the spacing between flange 30 and flange 52 is so pre-selected or adjusted that the flange 52 is positioned to apply force directly onto the inlet end 8 or the plug 12, plugging the inlet port 9, along the longitudinal axis 21.

The exemplary embodiments of FIGS. 1-10 and 18-21 may be also used in securely holding a pair of interconnected items together, for example such as interconnected electrical cords, so as to prevent the same from becoming unintentionally detached.

The chosen exemplary embodiments of the claimed invention have been described and illustrated for practical purposes so as to enable any person skilled in the art to which it pertains to make and use the same. It is therefore intended that all matters in the foregoing description and shown in the accompanying drawings be interpreted as illustrative and not in a limiting sense. It will be understood that variations, modifications, equivalents and substitutions for components of the specifically described exemplary embodiments of the invention may be made by those skilled in the art without departing from the spirit and scope of the invention as set forth in the appended claims.

Any element in a claim that does not explicitly state “means for” performing a specified function, or “step for” performing a specified function, is not to be interpreted as a “means” or “step” clause as specified in 35 U.S.C. §112, ¶6. In particular, any use of “step of” in the c aims is not intended to invoke the provision of 35 U.S.C. §112, ¶6.

Furthermore, the Abstract is not intended to be limiting as to the scope of the claimed invention and is for the purpose of quickly determining the nature of the claimed invention.

What is claimed is:

1. A locking device for a medical tube coupling, comprising:
(a) an elongated body defining a longitudinal axis of said device and positioned, during use of said locking device, adjacent an exterior surface of the medical tube coupling;
(b) a first flange at one end of said elongated body and extending therefrom, said first flange having a pair of surfaces being spaced apart from each other to define a thickness of said first flange, and a slot formed through said thickness in an open communication with and edge surface of said first flange; and
(c) a second flange at a longitudinally opposite end of said elongated body and extending therefrom.

2. The locking device of claim 1, further comprising a third flange disposed on said elongated body between said first and second flanges.

3. The locking device of claim 1, wherein said slot formed through said first flange includes an irregular inner surface, being flexible to accommodate tubes of varying exterior diameters.

4. The locking device of claim 1, wherein said slot includes a passageway being in an open communication with an edge surface of said first flange and having a pair of inwardly curved surface portions and further includes a cavity being in an open communication with said passageway and having outwardly curved surface portion(s), whereby said slot is configured and operable to accommodate medical tubes of varying external diameters.

5. The locking device of claim 1, wherein said second flange includes another a slot formed through a thickness thereof, said thickness defined by a pair of surfaces being spaced apart from each other.

6. A locking device for a medical tube coupling, comprising:
(a) an elongated body defining a longitudinal axis of said device;
(b) a first flange at one end of said elongated body and extending therefrom, said first flange having a pair of surfaces being spaced apart from each other to define a thickness of said first flange, and a slot formed through said thickness in an open communication with an edge surface of said first flange;
(c) a second flange at a longitudinally opposite end of elongated body; and
(d) a third flange disposed between said first and second flanges and having a pair of surfaces being spaced apart from each other to define a thickness of said third flange.

7. The locking device of claim 6, wherein said third flange has a rigid connection with an exterior surface of said elongated body and extends in a direction being different to a direction of said first flange, and wherein said second flange includes a pair of slots, one of said pair of slots is aligned with said slot in said first flange along a length of said elongated body and wherein an opposite one of said pair of slots is aligned with said third flange along said length of said elongated body.

8. The locking device of claim 6, further comprising:
(a) a pivotal or a rotational connection between said third flange and an exterior surface of said elongated body, whereby said third flange is extendable in a first direction being a direction of said first flange or in a second direction being different than said first direction; and
(b) a pair of spaced apart projections on said exterior surface of said elongated body caging said thickness of said third flange and preventing a movement thereof along a length of said elongated body.

9. The locking device of claim 6, further comprising a hollow sleeve, wherein said elongated body is disposed within said hollow sleeve so that said hollow sleeve pivots or rotates about said elongated body and wherein said third flange is attached to an exterior surface of said sleeve for a pivoting or a rotation therewith between a first direction being said direction of said first and second flanges and a second direction being different than said first direction.

10. The locking device of claim 6, wherein said first flange and/or said second flange are connected to said elongated body in one of a rigid and a rotational manner.

11. A locking device for a medical tube coupling, comprising:
(a) a first body defining a longitudinal axis of said device and including a first flange carried on one end of said body, said first flange having a pair of surfaces being spaced apart from each other to define a thickness of said first flange, and a slot formed through said thickness in an open communication with an edge surface of said first flange;
(b) a second body disposed along said longitudinal axis, said second body including a second flange carried on one end of said second body, said second flange having a pair of surfaces being spaced apart from each other to define a thickness of said second flange, and a slot formed through said thickness of said second flange in an open communication with an edge surface thereof; and
(c) a means for moving said first and second flanges toward or apart from each other.

12. The locking device of claim 11, wherein said means for moving said first and second flanges toward or apart from each other comprises:
(a) a hollow interior provided in one of said first and second bodies;
(b) an axially opposite end of an opposite one of said first and second bodies sized and shaped for a reciprocal linear movement within said hollow interior; and
(c) a spring disposed within said hollow interior.

13. The locking device of claim 11, wherein said means for moving said first and second flanges toward or apart from each other comprises:
(a) a bore provided in one of said first and second bodies;
(b) a first thread formed on an interior surface of said bore adjacent a free end of said one of said first and second bodies; and
(c) a second thread formed on an exterior surface of an axially opposite one of said first and second bodies.

14. The locking device of claim 13, wherein said one of said first and second bodies includes a portion carrying said bore that pivots in a relationship to a remaining portion of said one of said first and second bodies.

15. A locking device for a medical tube coupling, comprising:
(a) an elongated body defining a longitudinal axis of said device;
(b) a first flange having a pair of surfaces being spaced apart from each other to define a thickness of said first flange, and a slot formed through said thickness in an open communication with an edge surface of said first flange;
(c) a second flange having a pair of surfaces being spaced apart from each other to define a thickness of said second flange, said second flange further having a slot formed through said thickness of said second flange in an open communication with an edge surface thereof; and
(d) a pivotal or a rotational connection between one or both flanges and respective end(s) of said elongated body.

16. The locking device of claim 15, wherein said pivotal or rotational connection includes a bore in said respective end of said elongated body, and a stem upstanding on one of said pair of surfaces, said stem being sized and shaped to be received within said bore.

17. The locking device of claim 15, wherein said pivotal or rotational connection includes an aperture formed through said thickness of said first or second flange, a stem extending from said respective end of said elongated body, said stem sized and shaped to be received through said aperture.

18. The locking device of claim 15, further comprising a third flange carried on said elongated body between said first and second flanges and extending in a direction being different than said direction of said first and second flanges, said third flange having a pair of surfaces being spaced apart from each other to define a thickness thereof.

19. The locking device of claim 15, further comprising a third flange disposed between said first and second flanges and having a pair of surfaces being spaced apart from each other to define a thickness of said third flange, a pivotal or a rotational connection between said third flange and an exterior surface of said elongated body, whereby said third flange is extendable in a first direction being said direction of said first and second flanges or in a second direction being different than said first direction.

20. The locking device of claim 19, wherein said pivotal or rotational connection includes an aperture formed through said thickness of said third flange and receiving said exterior surface of said elongated body therethrough and a pair of spaced apart projections on an exterior surface of said elongated body caging said thickness of said third flange and preventing a movement thereof along a length of said elongated body.
